SEQUENCE LISTING (17 sequences)

SEQ ID NO: 1 - protein, 467 aa, Streptococcus suis
QDINQYFQSLTYEPQEILTNEGEYIDNPPATTGMLENGRFVVLRREKKNITNNSADIAVIDAKAANIYPGALLRADQNLLDNNPTLISIARGDLTLSLNLPGLANGDSHTVVNSPTRSTVRTGVNNLLSKWNNTYAGEYGNTQAELQYDETMAYSMSQLKTKFGTSFEKIAVPLDINFDAVNSGEKQVQIVNFKQIYYTVSVDEPESPSKLFAEGTTVEDLKRNGITDEVPPVYVSSVSYGRSMFIKLETSSRSTQVQAAFKAAIKGVDISGNAEYQDILKNTSFSAYIFGGDAGSAATVVSGNIETLKKIIEEGARYGKLNPGVPISYSTNFVKDNRPAQILSNSEYIETTSTVHNSSALTLDHSGAYVAKYNITWEEVSYNEAGEEVWEPKAWDKNGVNLTSHWSETIQIPGNARNLHVNIQECTGLAWEWWRTVYDKDLPLVGQRKITIWGTTLYPQYADEVIE

SEQ ID NO: 2 - DNA, 1401 bp, Streptococcus suis
caagatattaatcagtattttcaaagcttgacttacgagccacaagagattcttacaaatgagggagaatacattgataatccgccagcaacaactggtatgttagaaaacggacgttttgtagtacttcgcagagaaaagaagaatattacgaacaatagtgcagatattgctgttattgatgctaaggctgcaaatatttatccaggtgctttattgcgtgctgaccaaaatcttctggataataatccaacgcttatcagtattgcgcggggagatctgacgcttagtttgaatttacctggtttggccaatggggatagccacactgttgtaaattctccaacaagaagtactgttcgaacaggggtgaataaccttctgtctaaatggaataatacgtatgctggagagtatggcaatacccaagcagagcttcaatatgatgaaacaatggcatacagtatgtcacaattgaaaacgaagttcggaacctcttttgaaaaaattgctgtaccattagatatcaattttgatgccgtgaattcgggtgaaaaacaggttcagattgttaactttaaacaaatttattatacagttagtgttgatgaaccagaatctccaagcaagctttttgcagaagggacaactgtagaagatttgaaacgaaatgggataacagatgaggtacctcctgtttatgtttccagcgtttcttatggacgctctatgttcatcaagttagaaactagcagtaggagtacccaagttcaagccgcatttaaagcagccatcaaaggcgttgatattagtggcaatgctgagtatcaagacattctgaaaaatacttcattctctgcttatatttttggtggggatgcaggtagcgcggctactgttgtgagcggaaatattgaaacactgaagaagattattgaagaaggtgcaagatacggaaaactcaatccaggtgttccgatttcgtattcaaccaactttgtcaaagacaatagacctgctcagattttgagcaattcagagtacatagaaacaacttcaacagtccataatagcagtgcattgacattggatcattcaggtgcttatgttgcgaaatacaacattacttgggaagaagtatcttacaatgaagctggagaagaagtttgggaaccaaaagcttgggataagaatggtgtaaatctgacctcacactggagtgaaaccattcaaattccaggaaatgctcgcaatcttcatgtcaatattcaagaatgtacaggattagcatgggagtggtggagaacagtttatgacaaagatttaccacttgttggtcaacgtaaaataaccatctggggaacaacgttatacccacagtatgcggatgaggtgatagag

SEQ ID NO: 3 - protein, 499 aa, Streptococcus intermedius
ETPTKPKAAQTEKKTEKKPENSNSEAAKKALNDYIWGLQYDKLNILTHQGEKLKNHSSREAFHRPGEYVVIEKKKQSISNATSKLSVSSANDDRIFPGALLKADQSLLENLPTLIPVNRGKTTISVNLPGLKNGESNLTVENPSNSTVRTAVNNLVEKWIQNYSKTHAVPARMQYESISAQSMSQLQAKFGADFSKVGAPLNVDFSSVHKGEKQVFIANFRQVYYTASVDSPNSPSALFGSGITPTDLINRGVNSKTPPVYVSNVSYGRAMYVKFETTSKSTKVQAAIDAVVKGAKLKAGTEYENILKNTKITAVVLGGNPGEASKVITGNIDTLKDLIQKGSNFSAQSPAVPISYTTSFVKDNSIATIQNNTDYIETKVTSYKDGALTLNHDGAFVARFYVYWEELGHDADGYETIRSRSWSGNGYNRGAHYSTTLRFKGNVRNIRVKVLGATGLAWEPWRLIYSKNDLPLVPQRNISTWGTTLHPQFEDKVVKDNTD

SEQ ID NO: 4 - DNA, 1497 bp, Streptococcus intermedius
gaaacacctaccaaaccaaaagcagctcaaacagagaaaaaaaccgaaaagaaaccggaaaacagcaactctgaagctgcaaaaaaagctctgaatgattatatttggggattgcagtatgataaactaaacattttaacacaccaaggtgaaaaattgaaaaaccactctagccgcgaggcatttcatcgcccaggtgagtatgttgttatcgaaaagaaaaaacaaagcatttcaaacgcaacatctaagttatctgtaagttcagcaaatgatgaccgcatcttcccaggtgcattgctaaaagcagaccaaagtttgttagaaaatcttccaaccctaatcccagttaatcgcggcaaaacaactattagtgtaaacttaccgggattgaaaaatggcgaaagtaatcttacagttgaaaatccatccaacagtacagttcgaacagctgttaacaatttagttgaaaaatggattcaaaactactctaaaactcatgctgtaccagctagaatgcaatatgaatctattagcgcccaaagcatgagccaattacaagcaaaatttggtgctgatttctcaaaagtcggtgcaccacttaatgttgacttctcatctgttcacaaaggtgaaaaacaagtatttattgcgaacttcagacaagtttactacacagctagcgtagactctccaaatagtccttctgcactctttggctctggtataacaccaaccgatttaatcaatcgtggagttaattctaaaaccccaccagtttatgtttcaaatgtatcatatggccgtgcaatgtatgtgaaatttgaaactacaagcaagagtacaaaagtacaagccgctattgatgctgttgttaaaggagcaaaacttaaagctggaacagaatatgaaaacattctaaaaaatactaaaatcactgctgttgttctcggtggtaacccaggtgaagcttctaaagtcatcacaggtaatattgatactttgaaagatttgatccaaaaaggtagcaatttcagtgctcaaagtccagctgtaccaatctcttacactacttcttttgtgaaagacaattctattgcaactatccaaaacaacacagactacatcgaaacaaaagtaacatcctataaagatggtgctctcaccctcaatcatgatggtgctttcgttgcacgcttctatgtttattgggaagaactcggacatgatgctgatggctacgaaactattcgctcaagatcttggagtggaaatggctacaatcgcggtgcacactattctacaactctccgtttcaagggaaatgttagaaacattcgcgtaaaagtactaggagccactggactagcttgggagccttggagactgatctatagcaagaacgatcttcctttggttccacaaagaaacattagcacttggggaacaacccttcatccacagtttgaagataaagttgtgaaagataacactgat

SEQ ID NO: 5 - protein, 467 aa, Streptococcus suis
  MUTAGEN (22)..(22)
  MUTAGEN (157)..(157)
  MUTAGEN (426)..(426)
QDINQYFQSLTYEPQEILTNECEYIDNPPATTGMLENGRFVVLRREKKNITNNSADIAVIDAKAANIYPGALLRADQNLLDNNPTLISIARGDLTLSLNLPGLANGDSHTVVNSPTRSTVRTGVNNLLSKWNNTYAGEYGNTQAELQYDETMAYSMCQLKTKFGTSFEKIAVPLDINFDAVNSGEKQVQIVNFKQIYYTVSVDEPESPSKLFAEGTTVEDLKRNGITDEVPPVYVSSVSYGRSMFIKLETSSRSTQVQAAFKAAIKGVDISGNAEYQDILKNTSFSAYIFGGDAGSAATVVSGNIETLKKIIEEGARYGKLNPGVPISYSTNFVKDNRPAQILSNSEYIETTSTVHNSSALTLDHSGAYVAKYNITWEEVSYNEAGEEVWEPKAWDKNGVNLTSHWSETIQIPGNARNLHVNIQEATGLAWEWWRTVYDKDLPLVGQRKITIWGTTLYPQYADEVIE

SEQ ID NO: 6 - protein, 58 aa, Staphylococcus aureus
DNKFNKEQQNAFYEILHLPNLNEEQRNGFIQSLKDDPSQSANLLAEAKKLNDAQAPKA

SEQ ID NO: 7 - protein, 536 aa, Artificial Sequence
  Modified CDC comprising Hexa His-tag (1..9), Z-domain of protein A derived from S. aureus i10..67j, domains 1-3 of SLY (70..427), and domain 4 of SLY (428..536)
MKHHHHHHQDNKFNKEQQNAFYEILHLPNLNEEQRNGFIQSLKDDPSQSANLLAEAKKLNDAQAPKAGSQDINQYFQSLTYEPQEILTNECEYIDNPPATTGMLENGRFVVLRREKKNITNNSADIAVIDAKAANIYPGALLRADQNLLDNNPTLISIARGDLTLSLNLPGLANGDSHTVVNSPTRSTVRTGVNNLLSKWNNTYAGEYGNTQAELQYDETMAYSMCQLKTKFGTSFEKIAVPLDINFDAVNSGEKQVQIVNFKQIYYTVSVDEPESPSKLFAEGTTVEDLKRNGITDEVPPVYVSSVSYGRSMFIKLETSSRSTQVQAAFKAAIKGVDISGNAEYQDILKNTSFSAYIFGGDAGSAATVVSGNIETLKKIIEEGARYGKLNPGVPISYSTNFVKDNRPAQILSNSEYIETTSTVHNSSALTLDHSGAYVAKYNITWEEVSYNEAGEEVWEPKAWDKNGVNLTSHWSETIQIPGNARNLHVNIQEATGLAWEWWRTVYDKDLPLVGQRKITIWGTTLYPQYADEVIE

SEQ ID NO: 8 - protein, 563 aa, Artificial Sequence
  Modified CDC comprising Hexa His-tag (1..9), Z-domain of protein A derived from S. aureus i10..67j, domains 1-3 of SLY (70..454), and domain 4 of ILY (455..536)
MKHHHHHHQDNKFNKEQQNAFYEILHLPNLNEEQRNGFIQSLKDDPSQSANLLAEAKKLNDAQAPKAGSETPTKPKAAQTEKKTEKKPENSNSEAAKKALNDYIWGLQYDKLNILTHQCEKLKNHSSREAFHRPGEYVVIEKKKQSISNATSKLSVSSANDDRIFPGALLKADQSLLENLPTLIPVNRGKTTISVNLPGLKNGESNLTVENPSNSTVRTAVNNLVEKWIQNYSKTHAVPARMQYESISAQSMCQLQAKFGADFSKVGAPLNVDFSSVHKGEKQVFIANFRQVYYTASVDSPNSPSALFGSGITPTDLINRGVNSKTPPVYVSNVSYGRAMYVKFETTSKSTKVQAAIDAVVKGAKLKAGTEYENILKNTKITAVVLGGNPGEASKVITGNIDTLKDLIQKGSNFSAQSPAVPISYTTSFVKDNSIATIQNNTDYIETKVTSYKDSALTLDHSGAYVAKYNITWEEVSYNEAGEEVWEPKAWDKNGVNLTSHWSETIQIPGNARNLHVNIQEATGLAWEWWRTVYDKDLPLVGQRKITIWGTTLYPQYADEVIE

SEQ ID NO: 9 - DNA, 25 bp, Artificial Sequence
  Primer
gataacaaattcaacaaagaacaac

SEQ ID NO: 10 - DNA, 35 bp, Artificial Sequence
  Primer
gcctgcagctagcaagcttttggtgcttgtgcatc

SEQ ID NO: 11 - protein, 9 aa, Artificial Sequence
  Hexa His-tag
MKHHHHHHQ

SEQ ID NO: 12 - DNA, 174 bp, Staphylococcus aureus
gataacaaattcaacaaagaacaacaaaatgctttctatgaaatcttacatttacctaacttaaacgaagaacaacgcaatggtttcatccaaagcttaaaagatgacccaagccaaagcgctaaccttttagcagaagctaaaaagctaaatgatgcacaagcaccaaaagct

SEQ ID NO: 13 - DNA, 26 bp, Artificial Sequence
  Primer
gcggatccagcttttggtgcttgtgc

SEQ ID NO: 14 - DNA, 23 bp, Artificial Sequence
  Primer
cgctgcagttactctatcacctc

SEQ ID NO: 15 - DNA, 25 bp, Artificial Sequence
  Primer
cgggatccgaaacacctaccaaacc

SEQ ID NO: 16 - DNA, 33 bp, Artificial Sequence
  Primer
caatgtcaatgcactatctttataggatgttac

SEQ ID NO: 17 - DNA, 30 bp, Artificial Sequence
  Primer
acatcctataaagatagtgcattgacattg

(19) United States
(12) Patent Application Publication
(10) Pub. No.: US 2014/0039163 A1
(43) Pub. Date: Feb. 6, 2014

(54) CHOLESTEROL-DEPENDENT CYTOLYSIN VARIANT AND USE THEREOF IN DDS

(71) Applicant: The University of Tokushima, Tokushima (JP)

(72) Inventors: Hideaki NAGAMUNE, Tokushima-shi (JP); Toshifumi TOMOYASU, Tokushima-shi (JP); Atsushi TABATA, Tokushima-shi (JP)

(21) Appl. No.: 14/020,536

(22) Filed: Sep. 6, 2013

Related U.S. Application Data
(63) Continuation-in-part of application No. PCT/JP2012/056203, filed on Mar. 9, 2012.

(30) Foreign Application Priority Data
Mar. 9, 2011 (JP) 2011-051295

(51) Int. Cl. C07K 16/18; C07K 14/315

(52) U.S. Cl. CPC C07K 16/18; C07K 14/315
USPC 530/387.3; 530/350

(57) ABSTRACT

The present invention provides a carrier capable of delivering a medical agent capsule encapsulating a medical agent to various target cells and tissues to cause the medical agent to be taken therein in an efficient and highly safe manner, and a drug delivery system using the carrier. The carrier has a cell or tissue specific antibody bound to an antibody binding domain of a cholesterol-dependent cytolysin variant of the present invention. The carrier can also have bound thereto, as a transport material, a functional cell or a cholesterol-containing microcapsule filled with a medicinal ingredient or a bioactive substance via domain 4 of the variant.

### TECHNICAL FIELD

The present invention relates to a cholesterol-dependent cytolysin variant. Furthermore, the present invention relates to a use of the variant as a drug carrier (DDS carrier) in a drug delivery system (DDS), and a DDS using the drug carrier.

### BACKGROUND ART

In recent years, from a standpoint of placing great value on QOL (Quality of Life) of cancer patients, studies are conducted throughout the world for alternative therapies such as immune cell therapies and cancer missile therapies etc., with respect to mainstream cancer therapies such as cancer ablative operations, systemic medication with anticancer agents, and radiation irradiation. Thus, there has been a desire for development of tools and therapeutic agents which act by specifically and efficiently targeting cancer cells while having small side effects. For example, there have been many studies for developing targeting technology using a monoclonal antibody or the like as a targeting molecule for cancer, and anticancer agents to be linked thereto. However, in reality, technologies for causing such targeting molecule to accumulate at a target cancer cell in large quantity are not sufficient and need improvement.

Furthermore, a number of viral infectious diseases such as AIDS and hepatitis, congenital hereditary diseases, and illness based on abnormal gene expression have become a major issue in recent years. For onset suppression and therapy of such illnesses, application of gene therapies by repairing a specific gene or suppressing gene expression through RNAi technique has become an important task for future medical services. Although nonspecific carriers such as atelocollagen are currently used for delivering a medical agent for RNAi, it will become essential to selectively deliver a gene expression regulating agent to a target cell, tissue, or organ for the general practical use of gene therapy.

Due to such background, to date, there has been a demand to establish a drug delivery system (DDS) for treating an illness by delivering, to a target cell or tissue in an efficient and highly safe manner, a liposome or the like encapsulating a gene therapeutic agent or a therapeutic agent for cancer such as an anticancer agent at high concentration.

Examples of Literatures known in the art disclosing a technology similar to the present invention prior to this application include Patent Literature 1, and Non-Patent Literature 1 and 2.

Patent Literature (PTL) 1 shows that a variant with a Cys residue at the N-terminal of the cell membrane binding domain from intermedilysin, which specifically recognizes human cell membrane, has an ability to specifically bind to human cells. However, Patent Literature 1 merely suggests utilizing this characteristic for the use as a human cell membrane binding adapter. Although the human cell membrane binding adapter is useful for binding and fixing various substances to the human cell membrane, the adapter does not have a function of causing a substance to be taken into a cell and to be released therein.

Furthermore, Non-Patent Literature (NPL) 1 shows that, when a carcinoembryonic antigen (CEA) antibody is further connected to domain 4 of intermedilysin obtained by introducing thereto a Cys residue to the N-terminal of the cell membrane binding domain using the technology disclosed in Patent Literature 1 described above, it is possible to target cancer cells such as CEA positive human thyroid medullary cancer cells and the like, allowing application thereof to cancer therapy. Similar to Patent Literature 1 above, although this technology is useful in the sense that cancer cell can be targeted, the technology cannot be expected to provide an advantageous effect of causing a drug such as an anticancer agent to be taken into a cell and to be released therein.

Furthermore, Non-Patent Literature (NPL) 2 discloses a drug delivery system using cholesterol-dependent cytolysin (CDC), similar to the present invention. However, Non-Patent Literature 2 merely discloses a technology for transporting and delivering a drug specifically to a lung cancer cell by binding, to the N-terminal of a CDC variant (CDC-SS) having controlled membrane pore forming ability through introduction of an SS bond, a peptide (lung cancer targeting domain) having high affinity to lung cancer cells. Thus, the specificity is only for lung cancer cells, resulting in a problem of being less versatile due to lack of target flexibility.

### CITATION LIST

### Non-Patent Literature

NPL 1: H. Nagamune et al. (2004) Anticancer Res., Vol. 24(5), pp. 3367-3372.
NPL 2: “Development of an effective DDS for cancer therapy using a cancer targeting toxin” Presentation summaries of the 81st Annual Meeting of the Japanese Biochemical Society (Dec. 12, 2008).
NPL 3: Atsuyuki Tabata et al. (2009) Research report of Graduate School of Sociotechno Science University of Tokushima, No. 54, pp. 51-56.
NPL 4: H. Nagamune et al. (1996) Infect. Immun., Vol. 64(8), pp 3093-3100.
NPL 5: H. Nagamune et al. (2000) J. Clin. Microbiol., Vol. 38(1), pp 220-226.
NPL 6: H. Nagamune et al. (2004) Microbiol. Immunol., Vol. 48(9), pp. 677-692.
NPL 7: K. Ohkura et al. (2004) Anticancer Res., Vol. 24(5), pp. 3343-3354.
NPL 8: K. Sekiya et al. (2007) Microb. Infect., Vol. 9(11), pp. 1341-1350.
NPL 9: K. Semba et al. (1985) PNAS, Vol. 82(19), pp. 6497-6501
NPL 10: D. J. Slamon et al. (1987) Science, Vol. 235(4785), pp. 177-182.
NPL 11: I. Wiest et al. (2010) Anticancer Res., Vol. 30(5), pp. 1849-1853.
NPL 12: D. X. Zhou et al. (2011) Biochem. Biophys. Res. Commun. Vol. 405(2), pp. 325-332.
NPL 13: R. Li et al. (2004) Arch. Pathol. Lab. Med., Vol. 128(12), pp. 1412-1417.
NPL 14: S. Yonezawa and E. Satop (1997) Pathol. Int., Vol. 47(12), pp. 813-830.
NPL 15: E. Lacunza et al. (2010) Cancer Genet. Cytogenet., Vol. 201(2), pp. 102-120.

### SUMMARY OF INVENTION

### Technical Problem

An objective of the present invention is to solve the above described demand from the medical industry and the society, and the task is to provide: a carrier (drug carrier) capable of delivering, to a target cell and a tissue in an efficient and highly safe manner, a medical agent capsule encapsulating a medical agent; and a drug delivery system (DDS) using the carrier. Further preferably, an objective is to provide a carrier (drug carrier) that is efficient and highly safe, and can be used in all purposes for cancer therapy and gene therapy, and a drug delivery system (DDS) using the carrier.

### Solution to Problem

The present inventors have conducted thorough research to solve the above described task and have focused on cholesterol-dependent cytolysin (herein after, also abbreviated simply as “CDC”) that specifically binds to cell membranes or membranes containing cholesterol (CHL) and forms membrane pores (holes). The present inventors have discovered that, by using an antibody bound CDC variant created by modifying CDC in a manner described in the following (1) to (3), and causing an antibody binding domain located at the N-terminal region of the CDC variant to bind with a cell specific antibody or a tissue specific antibody, it is possible to bind thereto a CHL-containing liposome or the like encapsulating a desired medical agent. Furthermore, the present inventors have also discovered that, as a result, it is possible to selectively, efficiently, and safely deliver the medical agent-containing liposome to a target site recognized by the antibody for binding, and allow the function of the medical agent to be exerted accurately at the target site. With this, the present inventors have confirmed that the antibody bound CDC variant can effectively function as a drug carrier.

(1) An antibody binding domain of an antibody binding protein is bound to the N-terminal side of CDC.

In FIG. 1, the antibody binding domain is represented with reference character “Z”, and CDC is represented with reference characters 1 to 4. As shown in FIG. 1, the antibody binding domain (Z) is bound to the N-terminal side of domain 1 of CDC consisting of domains 1 to 4.

(2) In order to ensure stability of domain 4 (reference character “4” in FIG. 1) involved in membrane binding of CDC, a Cys residue in domain 4 is substituted with other stabilizing amino acids (e.g., Ala residue, etc.).

(3) At least two arbitrary positions in domains 1 to 3 (reference characters 1 to 3 in FIG. 1) involved in self-association and membrane penetration (membrane pore formation) of CDC are mutated to Cys to form an SS bond.

As described above, with the CDC variant (hereinafter, also referred to as “Z-CDC-SS”) modified as described in (1) to (3) above, it is possible to bind an antibody (IgG) that is tissue specific or cell specific with different antigenic specificity to the antibody binding domain (Z) located at its N-terminal region, and bind thereto a CHL-containing liposome (indicated with reference character “M” in FIG. 1) encapsulating a medical agent via domain 4. As a result, for example, when an antibody that selectively recognizes a protein specifically expressed in a cancer cell is bound to the antibody binding domain (Z) located at the N-terminal region and when a CHL-containing liposome encapsulating an anticancer agent is bound to domain 4, the anticancer agent (the CHL-containing liposome encapsulating the anticancer agent) can be transported specifically to the cancer cell or a cancer tissue identified as a target site. Furthermore, with regard to the CDC variant, its domains 1 to 3 are crosslinked through an SS bond, and the membrane penetration (membrane pore formation) function of the CDC is designed to be exerted only under reducing environment in which the SS bond is cleaved (e.g., cf. lower part of FIG. 2). Specifically, for example, the anticancer agent (CHL-containing liposome encapsulating the anticancer agent) is transported to the target site (cancer cell or cancer tissue) to be taken into the cancer cell through endocytosis, and a pore opens on the liposomal membrane encapsulating the anticancer agent only when it is exposed to a reducing environment generated by intracellular reduced glutathione or the like, resulting in intracellular release of the anticancer agent inside the liposomal membrane and exertion of anticancer activity (membrane pore formation and content release within a phagolysosome) (cf. FIG. 3).

In such manner, since the CDC variant (Z-CDC-SS) of the present invention can allow antibodies having various specificity to be linked to the antibody binding domain (Z) located at the N-terminal region, the CDC variant is widely applicable in a DDS as an all-purpose DDS carrier that targets various cells including cancer cells and virally infected cells. In addition, with the DDS carrier (antibody bound CDC variant) of the present invention obtained by binding a cell or tissue specific antibody to the CDC variant (Z-CDC-SS), it becomes possible to not only selectively deliver a drug (CHL-containing liposome encapsulating the drug) to a target cell of an antibody, but also reductively cleave the SS bond in the CDC variant upon intracellular intake of the drug after the delivery, thereby creating a hole in the CHL-containing liposome encapsulating the drug and releasing the drug within the target cell. As a result, medicinal effect of the drug can be exerted selectively and efficiently on the target cell. Therefore, an effective and safe therapeutic effect can be obtained with less effect to other cells and with small quantity of the drug.

The present invention has been accomplished based the findings above, and includes the following embodiments.

(I) Cholesterol-Dependent Cytolysin Variant (CDC Variant)

(i-1) A CDC variant comprising:

(1) an antibody binding domain;

(2) modified domains 1 to 3 wherein at least two arbitrary amino acid residues in domains 1 to 3 of cholesterol-dependent cytolysin are substituted with Cys residues to form an SS bond with each other under nonreducing condition; and

(3) a modified domain 4 wherein all Cys residues in domain 4 of cholesterol-dependent cytolysin are substituted with an amino acid residue selected from the group consisting of Ala, Ser, Gly, and Thr, wherein

the CDC variant has an ability to bind with a cell membrane or a cholesterol-containing liposomal membrane, and exerts an ability to form a membrane pore when an SS bond is cleaved under reducing condition.

(I-2) The CDC variant set forth in (I-1), wherein the CDC is at least one type selected from suilysin (SLY) and intermedilysin (ILY).

(I-3) The CDC variant set forth in (I-1) or (I-2), wherein domains 1 to 3 and domain 4 of the CDC are all derived from suilysin (SLY), or domains 1 to 3 of the CDC are derived from intermedilysin (ILY) and domain 4 of the CDC is derived from suilysin (SLY).

(I-4) The CDC variant set forth in any one of (I-1) to (I-3), wherein the antibody binding domain is Z-domain of Protein

A from Staphylococcus aureus, or B-domain of Protein G derived from a group G streptococci species.

(I-5) The CDC variant set forth in any one of (I-1) to (I-4), wherein the reducing condition is a reducing environment generated by intracellular glutathione.

(I-6) The CDC variant set forth in (I-5), wherein the reducing environment generated by the intracellular glutathione is an environment within a cytoplasm or phagolysosome containing reduced glutathione in an order of several mM, such as 1 to 10 mM, or 2 to 5 mM.

(II) Drug Carrier

(II-1) A drug carrier comprising the CDC variant set forth in any one of (I-1) to (I-6) and a cell or tissue specific antibody bound thereto via the antibody binding domain.

(II-2) The drug carrier set forth in (II-1), wherein the cell or tissue specific antibody is an antibody specifically recognizing an antigen such as proteins and oligosaccharides specifically expressed in a cancer cell, an antibody specifically recognizing a viral protein that appears on a cell membrane of a virally infected cell, or an antibody specifically recognizing an CD antigen expressed on an immune cell.

(III) Drug Delivery System

(III-1) A drug delivery system comprising the CDC variant, set forth in any one of (I-1) to (I-6),

the CDC variant having bound thereto, via domain 4 thereof, a cholesterol-containing microcapsule filled with a medicinal ingredient or a bioactive substance, or a functional cell such as an immune cell or a recombinant cell,

the CDC variant having bound thereto, via the antibody binding domain, a cell or tissue specific antibody.

(III-2) The drug delivery system set forth in (III-1), wherein the cell or tissue specific antibody is an antibody recognizing an antigen such as proteins and oligosaccharides specifically expressed in a cancer cell, an antibody recognizing a viral protein that appears on a cell membrane of a virally infected cell, or an antibody recognizing an CD antigen expressed on an immune cell.

It should be noted that, the drug delivery system can be realized by binding, to the drug carrier set forth in (II-1) or (II-2) via domain 4 of the CDC variant, a cholesterol-containing microcapsule filled with a drug or a functional cell such as an immune cell or a recombinant cell.

(III-3) The drug delivery system set forth in (III-1) or (III-2), wherein the medicinal ingredient or bioactive substance is at least one selected from the group consisting of compounds, peptides, proteins (including antibodies), and nucleic acids (genes encoding bioactive peptides and proteins, vectors containing those, DNA/RNA hybrids or chimeric polynucleotides, siRNAs, antisense nucleic acids [including RNA, DNA, PNA, and complexes of those], and substances with a dominant negative effect), having medicinal effects or being bioactive.

### Advantageous Effects of Invention

With the present invention, it is possible to provide a drug delivery system (DDS) capable of selectively and efficiently delivering, to a desired target cell, a cholesterol-containing microcapsule filled with a medicinal ingredient or a bioactive substance, or cells such as an immune cell or a recombinant cell, and allowing actions thereof to be exerted at the delivered location.

A similar CDC variant developed hitherto is capable of targeting a lung cancer cell, and its use has been extremely limited (Non-Patent Literature (NPL) 3). When compared thereto, the CDC variant of the present invention is much more useful as a molecular tool for producing a drug carrier used in a DDS. More specifically, with the CDC variant of the present invention, a desired antibody can be freely selected and bound thereto in accordance with a target cell which is the objective. Therefore, various drug carriers with high tropism for the target cell which is the objective can be produced. As a result, with the CDC variant of the present invention, by using various antibodies depending on the objective such as type of disease or site, a drug carrier having a desired cell tropism, i.e., a drug carrier applicable for treating various illnesses, can be produced. In this sense, the CDC variant of the present invention can be used as a material that can be used widely in all purposes and is useful for producing a desired drug carrier having high tropism for the target cell.

In addition, since the drug carrier (antibody bound CDC variant) of the present invention comprising the CDC variant of the present invention and the desired antibody bound thereto has an ability to bind to a cell membrane or cholesterol, the drug carrier can specifically and effectively transport and deliver a cell or a drug encapsulated within a cholesterol-containing liposome to the desired target cell. In the drug delivery system (DDS) of the present invention using the drug carrier, the drug carrier (antibody bound CDC variant) supporting the drug moves through body fluid, reaches a target cell of the antibody, and is intracellularly taken in through endocytosis. As a result, since an SS bond of the CDC variant is reductively cleaved to create a hole in the membrane of liposome or cell encapsulating the drug, the drug can be released intracellularly. Therefore, medicinal effect of the drug can be exerted effectively, efficiently, and selectively against the target cell without affecting other cells and tissues.

### BRIEF DESCRIPTION OF DRAWINGS

FIG. 1 is a schematic diagram of a drug delivery system (DDS) whose DDS carrier (drug carrier) is a CDC variant of the present invention. In the DDS shown in FIG. 1, a CDC variant (Z-CDC-SS) formed from zones A and B corresponds to a drug carrier, and zone C indicated by reference character M bond to an anchoring site (domain 4) of the drug carrier (CDC variant) represents “transportation object” transported to a target site by the drug carrier. In the DDS, zone A (reference character Z) is an antibody binding domain (also referred to as “targeting part”) to which various antibodies (IgG) having different antigenic specificities can be bound; and zone B consists of modified domains 1 to 4 of CDC(CDC-SS), including an anchoring site (domain 4) to which a CHL-containing liposomal membrane or a cell membrane binds, and a membrane pore forming site that exerts a membrane pore forming ability when an SS bond is cleaved under reducing condition (also respectively referred to as “anchor and membrane pore formation part”). In addition, in the DDS, the zone shown with reference character C is the “transportation object” that is bound to the anchoring site (domain 4) of the drug carrier (CDC variant). Examples of the transportation object include: liposome encapsulating therein an anticancer agent, a bacterial toxin, or a gene therapy agent; activation-induced NK cells; macrophages; and Tc cells.

FIG. 2 is a schematic diagram comparing, between wild type CDC (upper row) and the CDC variant (Z-CDC-SS) (lower row), binding to a cell membrane, formation of an association, and membrane penetration (membrane pore formation) (however, in the schematic diagram, description for “Z-domain” of the CDC variant is omitted). When the wild type CDC binds to a cell membrane, three-dimensional conformation of domains 1 to 3 changes to cause membrane penetration (membrane pore formation) and allow exhibition of toxin activity. However, when the CDC variant (Z-CDC-SS) binds to a cell membrane, since an SS bond is not cleaved under nonreducing condition and the three-dimensional conformation of domains 1 to 3 are maintained, membrane penetration (membrane pore formation) does not occur. The SS bond is cleaved only when being under reducing condition to change the three-dimensional conformation of domains 1 to 3, causing membrane penetration (membrane pore formation) and allowing exhibition of toxin activity. Thus, this figure shows that, by using the CDC variant (Z-CDC-SS), it is possible to control the expression of toxin activity of the CDC using presence of reducing/nonreducing condition.

FIG. 3 is a schematic diagram showing a therapeutic method using the drug delivery system (DDS) of the present invention.

In FIG. 4, “A” shows the three-dimensional conformation of suilysin (SLY) which is one CDC, and “B” shows the three-dimensional conformation of intermedilysin (ILY).

FIG. 5 shows two examples corresponding to the CDC variant (Z-CDC-SS) of the present invention created in Example 1. In FIG. 5, “A” shows an amino acid sequence of a CDC variant (Z-SLY-SS(C/A)) including, from the N-terminal side thereof, Z-domain of Protein A from Staphylococcus aureus having a His tag, modified domains 1, 2, and 3 of SLY, and modified domain 4 of SLY. In FIG. 5, “B” shows an amino acid sequence of a CDC variant (Z-cSLY-SS(C/A)) including, from the N-terminal side thereof, Z-domain of Protein A from Staphylococcus aureus having a His tag, modified domains 1, 2, and 3 of ILY, and modified domain 4 of SLY. In the figure, a doublet-underline region indicates the His tag region for purification, a region (58 a.a.) underlined with a two-headed arrow (<->) indicates Z-domain derived from Protein A of S. aureus, a region sandwiched between box arrows indicates the toxin domain in each CDC, black arrow head (solid triangle) indicates a Cys residue introduced for introducing an SS bond, and a white arrow head (open triangle) indicates a point at which a Cys residue is substituted with an Ala residue to stabilize the molecule.

FIG. 6 shows a result of electrophoresis (SDS-PAGE) and CBB staining of a purified preparation of the CDC variants (Z-SLY-SS(C/A), Z-cSLY-SS(C/A)) of the present invention created in Example 1, performed for examining their purity. In the figure, “A” shows the result for Z-SLY-SS(C/A) (molecular weight: 59.7 kDa), and “B” shows the result for Z-cSLY-SS(C/A) (molecular weight: 62.5 kDa).

FIG. 7 shows the results (Example 2) of evaluating membrane pore forming ability through hemolysis activity under reducing condition and under nonreducing condition for the CDC variants (Z-SLY-SS(C/A), Z-cSLY-SS(C/A)) of the present invention created in Example 1. In the figure, A shows the result for Z-SLY-SS(C/A), and B shows the result for Z-cSLY-SS(C/A). In each figure, dots connected by a line show hemolysis activity (%) of a CDC variant under reducing condition with 10 mM DTT, and open squares connected by a line show hemolysis activity (%) of a CDC variant under nonreducing condition. The horizontal axes show concentrations (ng/ml) for each of the CDC variants.

FIG. 8 shows results of evaluating binding of the CDC variants (Z-SLY-SS(C/A), Z-cSLY-SS(C/A)) of the present invention created in Example 1 against human serum-derived IgG (Human IgG) and rabbit serum-derived IgG (Rabbit IgG). In the figure, “A” shows the result for Z-SLY-SS(C/A), and “B” shows the result for Z-cSLY-SS(C/A) (Example 3). In each figure, dots connected by a line show binding, to IgG, of the CDC variants (Z-SLY-SS(C/A), Z-cSLY-SS(C/A)) of the present invention, and open squares connected by a line show binding, to IgG, of CDC variants (LTBP-SLY-SS(C/A), LTBP-cSLY-SS(C/A)) obtained by having, instead of Z-domain at the N-terminal region of the CDC variant of the present invention, a peptide formed from 20 amino acid residues not having antibody binding ability. The vertical axes show absorbance (415 nm) for evaluating binding to IgG through ELISA assay. Furthermore, the horizontal axes show concentration (μg/ml) for each of the CDC variants.

FIG. 9A shows the result of treating cells with anti-CEA antibody—[Z-cSLY-SS(C/A)]—uranine encapsulating liposomes, and FIG. 9B shows the result of treating cells with uranine-encapsulating liposomes (Example 4). In each figure, “1” is an image of cells observed with a phase contrast microscope, “2” is an uranine fluorescence image observed with an inverted fluorescence microscope, and “Merge” (only in A) is an overlay of the images of 1 and 2. Black arrows indicate CEA positive human colon cancer cells (Lovo cells), and white arrows indicated human normal fibroblast (NB1RGB cells).

FIG. 10 shows the result measuring survival rate during the course of time in Example 5 for cancer-bearing nude mice intraperitoneally administered with “5-FU-supporting antibody-bound CDC variant” (group 1: PBS having suspended therein “+HepG2, +α-CEA/Zcdc(ss) LIPO”), “5-FU-supporting CDC variant” (group 2: PBS having suspended therein “+HepG2, +Zcdc(ss) LIPO”), or PBS (group 3: “+HepG2 (control)”), and healthy nude mice intraperitoneally administered with PBS (group 4: “−HepG2 (control)”).

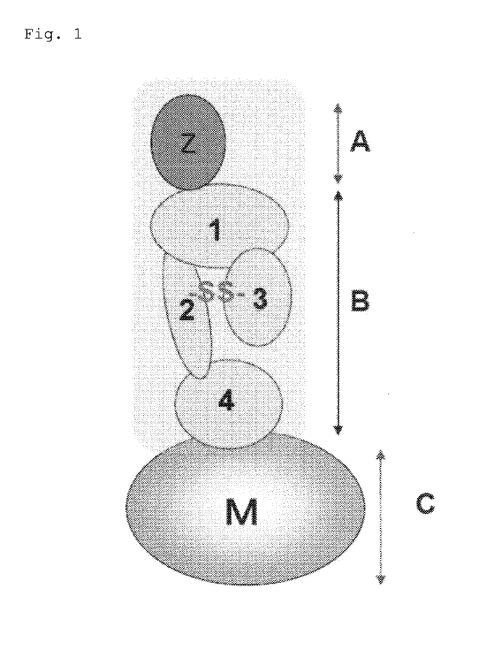

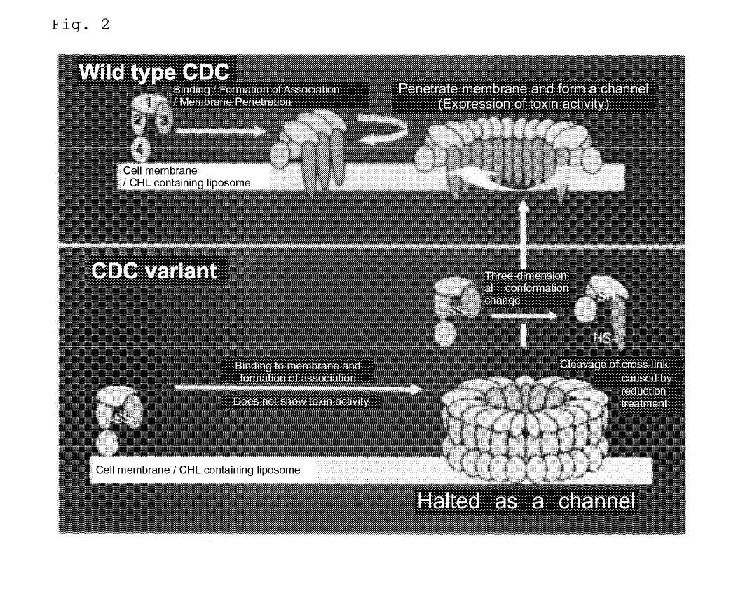

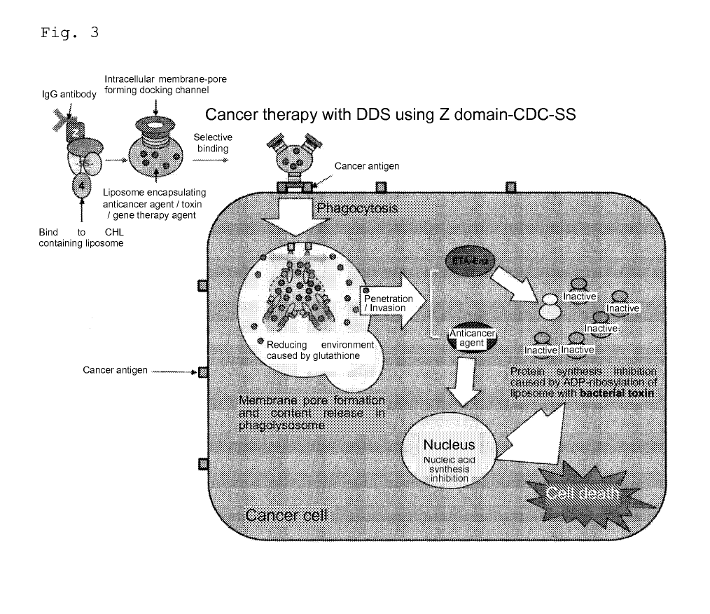

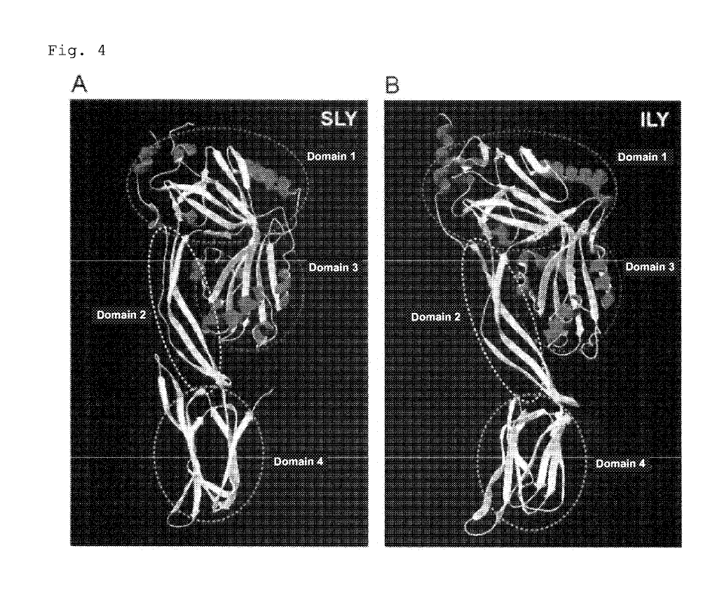

### DESCRIPTION OF EMBODIMENTS

(I) Cholesterol-Dependent Cytolysin Variant

A cholesterol-dependent cytolysin variant (CDC variant) which is the object of the present invention is a CDC variant that has domains shown in the following (1) to (3). The CDC variant has an ability to bind with a cell membrane or a cholesterol-containing liposomal membrane, and exerts an ability to form a membrane pore when an SS bond is cleaved under reducing condition.

(1) An antibody binding domain;

(2) modified domains 1 to 3 wherein at least two arbitrary amino acid residues in domains 1 to 3 of cholesterol-dependent cytolysin are substituted with Cys residues to form an SS bond with each other under nonreducing condition; and

(3) a modified domain 4 wherein all Cys residues in domain 4 of cholesterol-dependent cytolysin are substituted with an amino acid residue selected from the group consisting of Ala, Ser, Gly, and Thr.

In the present invention, cholesterol-dependent cytolysin (CDC) used for producing a CDC variant is a bacteria derived toxin that has an ability to bind to cell membranes of animals including mammals such as humans and others, and has a membrane pore forming ability of forming a pore on the cell membrane to which it has bound, causing a cell to perish as a result. Specific examples thereof include, but not limited to: suilysin (hereinafter, referred to as “SLY”) derived from Streptococcus suis; intermedilysin (hereinafter, referred to simply as “ILY”) derived from Streptococcus intermedius which is one species of anginosus group streptococci; streptolysin O (hereinafter, referred to as “SLO”) derived from group A hemolytic streptococcus (Streptococcus pyogenes); canilysin derived from Streptococcus canis; equisimilysin derived from Streptococcus dysgalactiae subsp. equisimilis; pneumolysin (hereinafter, referred to simply as “PLY”) derived from Streptococcus pneumoniae; perfringolysin O derived from Clostridium perfringens; tetanolysin O derived from Clostridium tetani; mitilysin (hereinafter, referred to simply as “MLY”), Streptococcus mitis derived human platelet aggregation factor (hereinafter, referred to simply as “Sm-hPAF”), and lectinolysin (hereinafter, referred to simply as “LLY”) which are three types of CDCs derived from Streptococcus mitis; pseudopneumolysin derived from Streptococcus pseudopneumoniae; vaginolysin (hereinafter, referred to simply as “VLY”) derived from Gardnerella vaginalis; listeriolysin O derived from Listeria monocytogenes; seeligeriolysin O derived from Listeria seeligeri; ivanolysin O derived from Listeria ivanovii; alveolysin O derived from Bacillus alvei; anthralysin O derived from Bacillus anthracis; and pyolysin O derived from Arcanobacterium pyogenes. Preferable CDCs include SLY and ILY, and, more preferably, CDC is SLY. In particular, when compared to other CDCs (e.g., ILY, Sm-hPAF, PLY, MLY, SLO, and VLY), SLY has markedly larger expression level (an yield close to a gram scale is obtained from 1L-culture scale) in an expression system using Escherichia coli, and its variants also result in high expression level. Therefore, SLY has an advantage of enabling cheap industrial production. Furthermore, when ILY is used as CDC, its domain 4 is preferably substituted with domain 4 of other CDCs (e.g., SLY or the like) capable of directly recognizing and binding to cholesterol. On the other hand, when domains 1 to 3 of ILY are used, it is possible to prepare modified domains 1 to 3 having high cleavage property in which the SS bond is cleaved under a reducing condition weaker than that for domains 1 to 3 of SLY. In that sense, ILY can be effectively used as CDC for producing the CDC variant of the present invention.

The structures and functions of these CDCs, particularly the structures and functions of ILY and SLO, are already published by the present inventors, and can be used as reference (cf. Non-Patent Literature 1, and 3 to 8).

Of the CDCs described above, except that Sm-hPAF and LLY have one additional domain at their N-terminals, all the CDCs have four domains including domains 1 to 3 involved in self-association and membrane penetration, and domain 4 (cell membrane binding domain) involved in binding with a cell membrane. The amino acid sequence of the full length of the mature form of SLY, and the base sequence encoding thereof are shown respectively in SEQ ID NOS: 1 and 2, and its three-dimensional conformation is shown in A of FIG. 4. Here, in the amino acid sequence of SLY set forth in SEQ ID NO: 1, a region of amino acid NOS. 1 to 358 corresponds to domains 1 to 3, and a region of amino acid NOS. 359 to 467 corresponds to domain 4.

The amino acid sequence of the full length of the mature form of ILY, and the base sequence encoding thereof are set forth respectively in SEQ ID NOS: 3 and 4, and its three-dimensional conformation is shown in B of FIG. 4. Here, in the amino acid sequence of ILY set forth in SEQ ID NO: 3, a region of amino acid NOS. 1 to 385 corresponds to domains 1 to 3, and a region of amino acid NOS. 386 to 499 corresponds to domain 4.

In the CDC variant of the present invention, the three-dimensional conformation of domains 1 to 3 is preferably maintained and fixed under nonreducing condition. Therefore, as described in (2) above, at least two arbitrary amino acid residues in domains 1 to 3 are substituted with Cys residues to form an SS bond with each other under nonreducing condition. The positions of the amino acid residues substituted with Cys residues are not particularly limited as long as the SS bond formed at the positions causes the three-dimensional conformation of domains 1 to 3 to be maintained under nonreducing condition, suppresses structural change associated with membrane pore formation, and does not compromise the membrane binding ability of domain 4 and the membrane pore forming ability of domains 1 to 3 of CDC under reducing condition. A preferable mode is one that is obtained by substituting an amino acid residue located at an arbitrary position of one domain among domains 1 to 3 and an amino acid residue located at an arbitrary position of another domain, each with a Cys residue, to form an SS bond between the Cys residues under nonreducing condition to link different domains of domains 1 to 3 for maintaining the three-dimensional conformation.

More specific examples of the regions having the amino acid residue that is to be substituted with a Cys residue include the regions shown in the following.

(1) Amino acid residues, one each from two three-dimensionally adjacent regions in domain 2 and domain 3, are mutated to Cys residues to introduce an SS bond. For example, in a case with ILY (SEQ ID NO: 3), one amino acid residue within a region of His48-Glu51, a region of Gln76-Thr82, or a region of Ile369-Val380 in domain 2 is substituted with a Cys residue, and one amino acid residue within a region of Gln181-Asp193 or a region of Val208-Glu212 in domain 3 is substituted with a Cys residue to form an SS bond. In a case with SLY (SEQ ID NO: 1), one amino acid residue within a region of Asn20-Glu23, a region of Lys48-Ser54, or a region of Ile342-Ser353 in domain 2 is substituted with a Cys residue, and one amino acid residue within a region of Tyr154-Ser166 or a region of Val181-Glu185 in domain 3 is substituted with a Cys residue to form an SS bond.

(2) Amino acid residues, one each from two three-dimensionally adjacent regions in a site in domain 1 and in two membrane penetration loop formation sites located in domain 3, are mutated to Cys residues to introduce an SS bond. For example, in a case with ILY (SEQ ID NO: 3), one amino acid residue within a region of Leu85-Asp92, a region of Leu108-Leu111, or a region of Ser346-Ile354 in domain 1 is substituted with a Cys residue, and one amino acid residue within a region of Phe190-Gly198 or a region of Val316-Gly322 in domain 3 is substituted with a Cys residue to form an SS bond. In a case with SLY (SEQ ID NO: 1), one amino acid residue within a region of Ile57-Ala64, a region of Leu80-Asn83, or a region of Gly319-Ile327 in domain 1 is substituted with a Cys residue, and one amino acid residue within a region of Phe163-Ala171 or a region of Ile289-Gly295 in domain 3 is substituted with a Cys to form an SS bond.

(3) Amino acid residues, one each from two three-dimensionally adjacent regions in a site in domain 3 having small three-dimensional conformation change and in two membrane penetration loop formation sites located in domain 3, are mutated to Cys residues to introduce an SS bond. For example, in a case with ILY (SEQ ID NO: 3), one amino acid residue within a region of Ile332-Gly342 in domain 3 is substituted with a Cys residue, and one amino acid residue within a region of Ile288-Lys293 or a region of Leu307-Ile312 in domain 3 is substituted with a Cys residue to form an SS bond. In a case with SLY (SEQ ID NO: 1), one amino acid residue within a region of Ile305-Gly315 in domain 3 is substituted with a Cys residue, and one amino acid residue within a region of Phe261-Lys266 or a region of Leu280-Phe285 in domain 3 is substituted with a Cys residue to form an SS bond.

(4) Amino acid residue, one each from two three-dimensionally adjacent regions in two membrane penetration loop formation sites located in domain 3, are mutated to Cys residues to introduce an SS bond. For example, in a case with ILY (SEQ ID NO: 3), one amino acid residue within a region of Arg269-Lys293, a region of Thr313-Val315, or a region of Ala324-Val327 in domain 3 is substituted with a Cys residue, and one amino acid residue within a region of Lys213-Gln222 or a region of Val197-Val203 in domain 3 is substituted with a Cys residue to form an SS bond. In a case with SLY (SEQ ID NO: 1), one amino acid residue within a region of Arg242-Lys266, a region of Ser286-Tyr288, or a region of Ala297-Val300 in domain 3 is substituted with a Cys residue, and one amino acid residue within a region of Lys186-Gln195 or a region of Ile170-Ile176 in domain 3 is substituted with a Cys residue to form an SS bond.

(5) Amino acid residues at two locations in one of or both of two membrane penetration loop formation sites located in domain 3 are mutated to Cys residues to introduce an SS bond, such that loop expansion caused by three-dimensional conformation change does not occur. For example, in a case with ILY (SEQ ID NO: 3): amino acid residues, one each from a region of Ile288-Leu297 and a region of Ile332-Gln340 in domain 3, are each substituted with a Cys residue to form an SS bond; or amino acid residues, one each from a region of Gln181-Phe190 and a region of Glu176-Ser179, are each substituted with a Cys residue to form an SS bond. In a case with SLY (SEQ ID NO: 1): amino acid residues, one each from a region of Phe261-Ile270 and a region of Ile305-Glu313 in domain 3, are each substituted with a Cys residue to form an SS bond; or amino acid residues, one each from a region of Tyr154-Phe163 and a region of Asp149-Met152, are each substituted with a Cys residue to form an SS bond.

A more preferable mode is a mode of (1), in which domains 2 and 3, which are adjacent to each other as shown in the three-dimensional conformation in FIGS. 4 A and B, are connected through formation of an SS bond under nonreducing condition between Cys residues located at the substituted positions in each of the domains.

The number of locations where an SS bond is formed is not limited to a single location, and is sufficient when the number of locations is one or more. The number of locations is preferably one or two, and more preferably one.

The SS bond formed in the regions of domains 1 to 3 is stably formed under nonreducing conditions, but is cleaved under reducing conditions. Examples of such reducing conditions include a reducing environment generated by intracellular glutathione. Specific examples of such environment include an environment within a cytoplasm or phagolysosome containing reduced glutathione in an order of, for example, several mM, such as 1 to 10 mM, or 2 to 5 mM.

When exposed to such a reducing condition, the SS bond formed in regions of domains 1 to 3 is cleaved, and a restraint caused by three-dimensional conformation is removed. Therefore, by having the three-dimensional conformation changed, the membrane pore forming ability that is originally possessed by domains 1 to 3 is exerted.

As described above, the CDC variant of the present invention allows ON/OFF control of the membrane pore forming ability of domains 1 to 3, by utilizing the SS bond formed in the regions of domains 1 to 3 under reducing conditions or nonreducing conditions. Therefore, the region of domain 4 in CDC not involved in ON/OFF of the membrane pore forming ability is preferably stabilized regardless of being under reducing conditions or nonreducing conditions as describe in (3) above, by substituting all Cys residues in domain 4 with an amino acid residue selected from the group consisting of Ala, Ser, Gly, and Thr. Since these amino acids of Ala, Ser, Gly, and Thr have common features such as having structures and physical properties similar to Cys, and being unlikely to naturally oxidize; any one of them can be selected.

For example, the Cys residue at position 426 in domain 4 of SLY (in the amino acid sequence set forth in SEQ ID NO: 1, the Cys residue at position 426) is preferably substituted with an Ala residue.

Furthermore, in the CDC variant of the present invention, the antibody binding domain is a domain to which antibodies (IgG) having different antigenic specificity can be bound. Specific examples thereof include Z-domain which is the antibody binding region of Protein A from Staphylococcus aureus. The amino acid sequence of the Z-domain is set forth in SEQ ID NO: 6. In addition, other than the Z-domain, as the antibody binding domain, B-domain which is the antibody binding region of Protein G derived from a group G streptococci species, and antibody binding regions of similar antibody binding proteins derived from other streptococci may also be used.

As long as the antibody binding domain's antibody (IgG) binding ability is not compromised, the antibody binding domain may have amino acid substitutions added for the purpose of reducing its antigenicity, or may have tags that are convenient for purification, such as, on the N-terminal side, a His tag, a FLAG tag, a myc tag, an antigen epitope tag, a glutathione-S-transferase tag, a maltose binding protein tag, etc. A hexa His tag having the amino acid sequence set forth in SEQ ID NO: 11 may be illustrated as one example of such tags. However, the tag is not limited thereto.

The CDC variant of the present invention described above has: (1) the antibody binding domain (hereinafter, also abbreviated as “Z”) which may include a tag at the N-terminal; (2) modified domains 1 to 3 obtained by substituting arbitrary amino acid residues in domains 1 to 3 of CDC with Cys residues to form an SS bond under nonreducing condition; and (3) a modified domain 4 obtained by substituting all Cys residues in domain 4 of CDC with an amino acid residue selected from the group consisting of Ala, Ser, Gly, and Thr (these are collectively represented as “X” instead of each of the amino acid's single letter notation). Therefore, the CDC is represented as “Z-CDC-SS(C/X)” for convenience (e.g., “Z-SLY-SS(C/X)” when the CDC is SLY, and “Z-SLO-SS(C/X)” when the CDC is SLO).

The CDC variant of the present invention has these domains of (1), (2), and (3) each in this order from the N-terminal side. However, as long as the function of the present invention is retained, these domains may be directly linked in this order, or may include one or more of any amino acid residues between each of the domains.

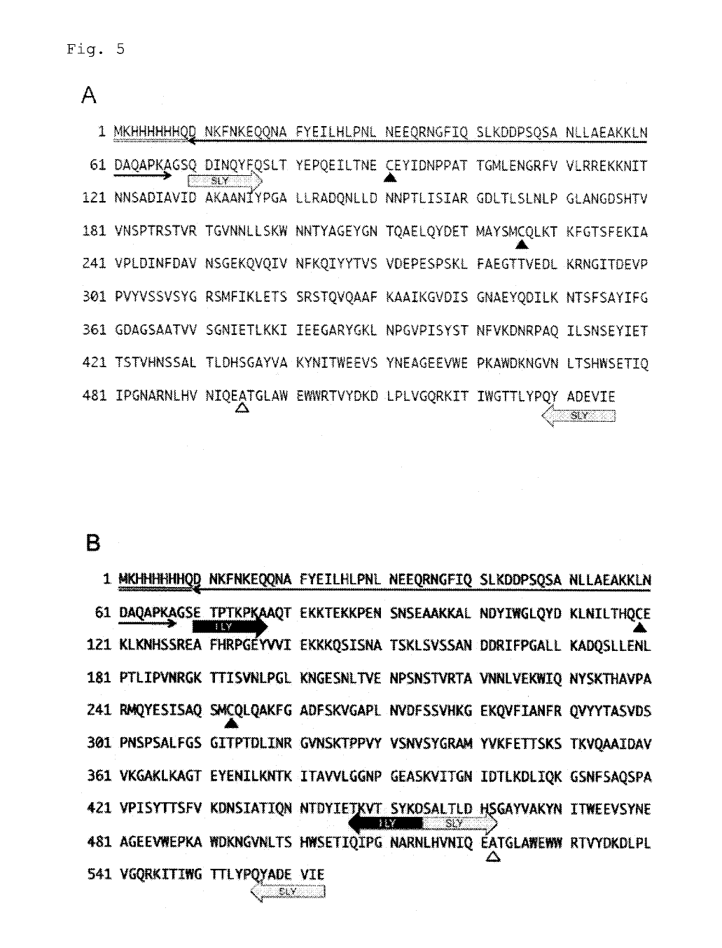

Examples of Z-SLY-SS(C/X) include Z-SLY-SS(C/A) (SEQ ID NO: 7, FIG. 5A) created in Example 1 described later. In the amino acid sequence of Z-SLY-SS(C/A), regions of amino acid NOS. 1 to 9 and NOS. 10 to 67 respectively correspond to the hexa His tag and the antibody binding region (Z-domain) of Protein A from Staphylococcus aureus, and these regions function as the antibody binding domain. Furthermore, in the amino acid sequence of Z-SLY-SS(C/A), regions of amino acid NOS. 70 to 427 and 428 to 536 respectively correspond to domains 1 to 3 and domain 4 of SLY. It should be noted that, in the antibody binding domain, the hexa His tag region (region of amino acid NOS. 1 to 9 in SEQ ID NO: 7) is optional, and the antibody binding region (Z-domain) (region of amino acid NOS. 10 to 67 in SEQ ID NO: 7) of Protein A from Staphylococcus aureus alone functions as the antibody binding domain.

In Z-CDC-SS(C/X), regions of domains 1 to 3 and domain 4 may all be derived from CDC of a single species, or may be derived from different CDCs. A preferable example of the CDC variant (Z-CDC-SS(C/X)) derived from CDC of a single species is a CDC variant whose regions of domains 1 to 3 and domain 4 are all derived from SLY (referred to as “Z-SLY-SS(C/X)”). Furthermore, a preferable example of the chimeric CDC variant (Z-cCDC-SS(C/X)) derived from CDCs from different species is a chimeric CDC variant whose regions of domains 1 to 3 are derived from ILY and domain 4 is derived from SLY (referred to as “Z-cSLY-SS(C/X)”).

Examples of Z-cSLY-SS(C/X) include Z-cSLY-SS(C/A) (SEQ ID NO: 8, FIG. 5B) created in Example 1 described later. In the amino acid sequence of Z-cSLY-SS(C/A), regions of amino acid NOS. 1 to 9 and NOS. 10 to 67 respectively correspond to the hexa His tag and the antibody binding region (Z-domain) of Protein A from Staphylococcus aureus, and these regions function as the antibody binding domain. Furthermore, in the amino acid sequence of Z-cSLY-SS(C/A), the region of amino acid NOS. 70 to 454 corresponds to domains 1 to 3 of ILY, and the region amino acid NOS. 455 to 563 corresponds to domain 4 of SLY. It should be noted that, in the antibody binding domain, the hexa His tag region (amino acid NOS. 1 to 9 in SEQ ID NO: 8) is optional, and the antibody binding region (Z-domain) (amino acid NOS. 10 to 67 in SEQ ID NO: 8) of Protein A from Staphylococcus aureus alone functions as the antibody binding domain.

(II) Drug Carrier

The drug carrier of the present invention comprises the CDC variant (Z-CDC-SS(C/X)) of the present invention having the antibody binding domain at the N-terminal region, and a cell specific antibody or a tissue specific antibody bound to the CDC variant via the antibody binding domain.

Examples of the cell specific antibody or the tissue specific antibody include: antibodies capable of specifically recognizing antigens such as protein and oligosaccharide structures that are specifically or over expressed in cancer cells or cancer tissues; antibodies capable of specifically recognizing viral proteins (antigens) that appear specifically on a cell membrane of a virally infected cell; and antibodies capable of specifically recognizing various CD antigens expressed specifically on the cell surface of immune cells.

Preferably, the antibody is a human antibody recognizing an antigen expressed specifically on a cancer cell or a cancer tissue. Here, examples of the human antibody include antibodies that are obtained from a hybrid between a variable region gene of an antibody of an animal other than human and a constant region gene of a human IgG; and antibodies produced through induction in a transgenic animal expressing a human antibody gene.

Examples of an antigen expressed specifically in cancer cells or cancer tissues include: carcinoembryonic antigen (CEA); HER2 expressed in, for example, breast cancer, salivary gland cancer, and ovarian cancer (Non-Patent Literature (NPL) 8 and 9); sialyl Lewis A (CA19-9) expressed in pancreatic cancer, biliary tract cancer, laryngeal cancer, gastric cancer, colon cancer, and the like (Non-Patent Literature (NPL) 10); HCA expressed in cholangiocellular cancer and the like (Non-Patent Literature (NPL) 11 and 12); and MUC-1 expressed in pancreatic cancer, breast cancer, and the like (Non-Patent Literature (NPL) 13 and 14).

Furthermore, examples of viral proteins (antigens) expressed specifically on a cell membrane of a virally infected cell include envelope proteins from envelope viruses such as HIV and hepatitis C virus.

Since the drug carrier of the present invention has on the C-terminal side thereof domain 4 (modified domain 4) of the CDC variant capable of binding to a cell membrane or cholesterol, it is possible to bind, to domain 4, a cell or a cholesterol (CHL)-containing microcapsule. Examples of the cell that is to be bound include, but not limited to, an immune cell activated as a cell-mediated medicine, and a cell having introduced therein a desired gene. In addition, examples of the CHL-containing microcapsule include, but not limited to, a microcapsule encapsulating a desired drug. With the drug carrier of the present invention having bound thereto these cells and CHL-containing microcapsules via domain 4, it is possible to transport a drug or a cell having desired activity or a gene to a target cell or tissue, and exert the function of the cell or drug at the target cell or tissue. Thus, the drug carrier of the present invention is useful as a drug carrier which is a tool for the drug delivery system (DDS) described later.

For example, when an antibody specifically recognizing an antigen such as a protein expressed specifically on a cancer cell or a cancer tissue is bound to the antibody binding domain of the drug carrier of the present invention, the drug carrier gains tropism for the cancer cell or the cancer tissue. By using this, it is possible to deliver a lethal anticancer agent selectively to the cancer cell or the cancer tissue and annihilate those, or deliver a gene therapy agent selectively to the cancer cell or the cancer tissue to be intracellularly taken therein for suppressing expression of mRNA involved in cancer.

Furthermore, when an antibody that specifically recognizes and binds to a viral protein expressed specifically on a cell membrane of a virally infected cell is bound to the drug carrier of the present invention via the antibody binding domain, the drug carrier gains tropism for the virally infected cell. By using this, it is possible to deliver a lethal medical agent selectively to the virally infected cell and annihilate the virally infected cell, or deliver a gene therapy agent selectively to the virally infected cell to be intracellularly taken therein for suppressing expression of mRNA of viral proteins or destroying or inactivating a viral gene inserted in the genome.

Furthermore, when an antibody that specifically recognizes and binds to one of various CD antigens expressed specifically on the cell surface of immune cells is bound to the drug carrier of the present invention via the antibody binding domain, the drug carrier gains immune cell tropism. By using this, it is possible to deliver a desired medical agent or gene therapy agent selectively to one of various immune cells.

(III) Drug Delivery System (DDS)

As described above, the DDS of the present invention utilizes the drug carrier of the present invention, and is obtained by binding, to domain 4 of the CDC variant of the drug carrier, a cholesterol-containing microcapsule encapsulating a medicinal ingredient or a bioactive substance (hereinafter, these are collectively referred to simply as “drug”), or cells (functional cells) having various useful functions.

There is no particular limitation in the cholesterol-containing microcapsule as long as it contains cholesterol as a structural component of the membrane, and a cholesterol-containing liposome is a suitable example thereof. In order to have domain 4 of the CDC variant recognize and bind to the cholesterol contained in the microcapsule, the concentration of the cholesterol contained in the microcapsule is preferably 30 mole % or higher. Although there is no limit in the concentration, examples thereof include 30 to 60 mole %, and more preferably 40 to 50 mole %.

Although the drug to be encapsulated in the cholesterol-containing microcapsule is not particularly limited, examples thereof include compounds, peptides, proteins (including antibodies), and nucleic acids (genes encoding bioactive peptides and proteins, vectors containing those, DNA/RNA hybrids or chimeric polynucleotides, siRNAs, antisense nucleic acids [including RNA, DNA, PNA, and complexes of those], and substances with a dominant negative effect), which are bioactive or have medicinal effects in vivo such as anticancer action, anti-inflammatory effect, anti-angiogenic action, antibacterial action, antiviral action, apoptosis induction action, gene expression inhibitory action, and gene expression inductive action.

These drugs may be encapsulated in the cholesterol-containing microcapsule together with pharmaceutically acceptable carriers and additives.

Examples of the functional cell include immune cells (NK cells, macrophages, etc.) having improved immune activity, and cells having introduced therein various compounds, peptides, proteins, or nucleic acids.

The drug delivery system of the present invention may be administered through various routes including both orally and parenteral routes, and examples of the administration route include, but not limited to, oral, intravenous, intramuscular, subcutaneous, local, rectal, intraarterial, intraportal, intraventricular, transmucosal, percutaneous, intranasal, intraperitoneal, intrapulmonary, and intrauterine routes. The drug delivery system may be formulated into a dosage form suitable for each of the administration routes. As the dosage form and formulating method, any of those known in the art may be selected as appropriate.

Examples of dosage forms suitable for oral administration include, but not limited to, powder, granule, tablet, capsule, liquid agent, suspension, emulsion, gel, and syrup. Examples of the dosage form suitable for parenteral administration include injections such as injectable solutions, injectable suspensions, injectable emulsions, and ready-to-use injections. The injections also include intravenous agents. Formulations for parenteral administration may be in a form such as an aqueous or non-aqueous isotonic sterile solution or suspension. Preferably, the formation is in a parenteral administration form.

The present invention also includes administration of the drug delivery system of the present invention containing an effective dose of the functional cell or the microcapsule encapsulating the drug to a subject in need for that. As described herein, an effective dose is an amount that reduces the onset of a target disease, relieves symptoms, or prevents progression; and is preferably an amount that prevents the onset of a target disease or heals a target disease. In addition, an effective dose is preferably an amount that does not cause adverse influences that outweigh the benefit by the administration. Such amount can be appropriately determined through in vitro tests using cultured cells, and tests using model animals such as mice, rats, dogs, or pigs. Such testing methods are well known by those skilled in the art.

The specific dosage of the drug delivery system administered in the present invention can be determined by taking into consideration various conditions regarding the subject in need for treatment, and examples thereof include severity of the symptom, the subject's general health condition, age, body weight, the subject's sex, diet, timing and frequency of administration, medication that is used in combination, reaction to therapy, and compliance to therapy. Examples of the administration frequency include, although it depends on the quality of the used drug or functional cell and the condition of the subject as described above: several times per day (i.e., 2, 3, 4, 5, or more times per day); once a day; once every several days (i.e., once every 2, 3, 4, 5, 6, 7 days etc.); once per week; and once every several weeks (i.e., once every 2, 3, 4 weeks etc.).

The subject that is the target of the drug delivery system of the present invention may be any living organism, and is preferably an animal, more preferably a mammal, and further preferably human. In the present invention, the subject may be healthy or may be affected with some sort of disease. However, in the case where treatment for a disease is to be planned, a subject typically refers to one that is affected with the same disease, or one having a risk of being affected.

The drug delivery system which is the object of the present invention includes a drug delivery system of the following modes.

(1) A drug delivery system for delivering an anticancer medical agent specifically to a cancer cell and performing efficient treatment. The drug delivery system is obtained by binding the CDC variant (Z-CDC-SS(C/X)) linked to an antibody against a cell surface marker molecule specific to one of various cancer cells, with a liposome encapsulating an anticancer agent and cancer-cell death inducing protein/toxin (anticancer medical agent), and is to be introduced in blood or to a local cancer tissue of a cancer patient.

(2) A drug delivery system, whose target is a virally infected cell expressing a viral antigen on the cell membrane, for effectively suppressing viral expression. The drug delivery system is obtained by binding the CDC variant (Z-CDC-SS(C/X)) of the present invention linked to an antibody that is specific against the viral antigen, with a liposome encapsulating an RNAi agent for selectively suppressing gene expression of the infected virus, or a fragment of DNA or RNA and a vector encoding those for specifically inactivating or destroying the viral genome. The drug delivery system is to be introduced in blood of an infected patient.

(3) A drug delivery system, whose target is cells forming a tissue/organ that has undergone an onset of an illness caused through expression of a specific responsible protein, for effectively treating the illness. The drug delivery system is obtained by binding the CDC variant linked to an antibody that recognizes an antigen specific to the tissue/organ, with a liposome encapsulating an RNAi agent for selectively suppressing expression of the responsible protein gene. The drug delivery system is to be introduced in blood or locally to the tissue/organ of the patient.

(4) A drug delivery system, whose target is cells including a tissue/organ that has undergone an onset of an illness caused through lack of expression of a specific responsible protein gene due to damage thereto, for effective therapy of the illness. The drug delivery system is obtained by binding the CDC variant linked to an antibody that recognizes an antigen specific to the tissue/organ, with a liposome encapsulating a gene fragment for substituting an abnormal gene of the responsible protein with a normal gene, or a gene therapy agent such as an expression vector for the normal protein of the responsible protein. The drug delivery system is to be introduced in blood or locally to the tissue/organ of the patient to conduct gene repair or gene complementation.

(5) A drug delivery system whose target is cells expressing a specific antigen on their cell surfaces. The drug delivery system is obtained by binding the CDC variant linked to an antibody against the cell surface antigen, with a liposome encapsulating a vector plasmid, a DNA fragment of a gene, siRNA for RNAi, a peptide, a protein, or a small molecular medical agent, for expressing or suppressing the specific gene. The drug delivery system is caused to take effect on those cells for effectively introducing the encapsulated substance in the cells.

### EXAMPLES

### Example 1

### Creation of CDC Variant (Z-CDC-SS) Expression System

### (1) Description of CDC Variant

The following variants were created using suilysin (hereinafter, referred to simply as “SLY”) and intermedilysin (hereinafter, referred to simply as “ILY”) which are cholesterol-dependent cytolysins (hereinafter, referred to simply as “CDC”). SLY is a CDC derived from Streptococcus suis and encoded by sly gene, and is a 52 kDa membrane pore forming protein that is activated or protected from inactivation by thiol. The amino acid sequence of the full length of its mature form, and the base sequence encoding thereof are set forth respectively in SEQ ID NOS: 1 and 2. In addition, ILY is a CDC derived from Streptococcus intermedius which is one species of anginosus group streptococci, encoded by ily gene, and is a 55 kDa membrane pore forming protein. The amino acid sequence of the full length of its mature form, and the base sequence encoding thereof are set forth respectively in SEQ ID NOS: 3 and 4. SLY and ILY both have four domains including domains 1 to 3 involved in self-association and membrane penetration, and domain 4 (cell membrane binding domain) involved in binding with a cell membrane.

(1-1) Description of Z-SLY-SS(C/A) (FIG. 5A, SEQ ID NO: 7)

A Cys residue (SEQ ID NO: 1: position 426) located in domain 4 of SLY was substituted (Cys426Ala) with Ala to improve stability. In addition, a Gly residue (SEQ ID NO: 1: position 22) located in domain 2 and a Ser residue (SEQ ID NO: 1: position 157) located in domain 3 were both point-mutated to Cys (Gly22Cys, Ser157Cys) to introduce an SS bond. As a result, three-dimensional conformation change of domains 1 to 3 was restrained, and improvement was made, enabling the toxin activity to be exerted only in reducing environment that cleaves an SS bond (creation of SLY-SS(C/A)). The amino acid sequence of the full length of SLY-SS(C/A) is set forth in SEQ ID NO: 5.

On the N-terminal side thereof, Z-domain (SEQ ID NO: 6) which is the antibody binding domain of Protein A from Staphylococcus aureus was fused (creation of Z-SLY-SS(C/A)). The amino acid sequence of the full length of Z-SLY-SS(C/A) is shown in A of FIG. 5 and SEQ ID NO: 7.

(1-2) Description of Z-cSLY-SS(C/A) (FIG. 5B, SEQ ID NO: 8)

A Gly residue (SEQ ID NO: 3: position 50) located in domain 2 and a Ser residue (SEQ ID NO: 3: position 184) in domain 3 of ILY were both point-mutated to Cys (Gly50Cys, Ser184Cys) to introduce an SS bond. As a result, three-dimensional conformation change of domains 1 to 3 of ILY was restrained, and improvement was made, enabling the toxin activity to be exerted only in reducing environment that cleaves an SS bond (creation of ILY-SS).

The regions of domains 1 to 3 (region of amino acid NOS. 1 to 358 in SEQ ID NO: 5) of SLY-SS(C/A) described in (1-1) were substituted with regions of domains 1 to 3 of ILY-SS created as described above to create a chimeric CDC variant (hereinafter, referred to as “cSLY-SS(C/A)” having regions of domains 1 to 3 of ILY-SS and domain 4 of SLY-SS(C/A)). Next, on the N-terminal side thereof, Z-domain (SEQ ID NO: 6) which is the antibody binding domain of Protein A from Staphylococcus aureus was fused (creation of Z-cSLY-SS(C/A)). The amino acid sequence of the full length of Z-cSLY-SS(C/A) is shown in B of FIG. 5 and SEQ ID NO: 8.

In the following, detailed description of the method for creating these CDC variants (Z-SLY-SS(C/A), Z-cSLY-SS(C/A)) will be provided.

(2) Method for Creating CDC Variants

### (2-1) Creation of Z-SLY-SS(C/A)

By using purified genome DNA of strain IFO12732 of Staphylococcus aureus as a template, the part of the gene having the Z-domain with the antibody binding ability in Protein A was amplified using the following primer set in PCR (35 cycles of 10 seconds at 98° C., 5 seconds at 55° C., and 15 seconds at 72° C.; followed by 5 minutes at 72° C.).

<Primer set>
SEQ ID NO: 9
5′-GATAACAAATTCAACAAAGAACAAC-3′:
SEQ ID NO: 10
5′-GCCTGCAGCTAGCAAGCTTTTGGTGCTTGTGCATC-3′:

Next, the obtained amplification fragment was cut with PstI, and the cut product was ligated within the PvuII and PstI sites of pQE-1 (Registered trademark of Qiagen) plasmid for 2 hours at 16° C. using DNA Ligation Kit (TaKaRa) to obtain a clone for expressing a product fused with a hexa His tag (MKHHHHHHQ: SEQ ID NO: 11) on the C-terminal side thereof.

Next, in order to add a BamHI site to the 3′ terminal side of the Z-domain gene (SEQ ID NO: 12), this plasmid was used as a template for amplification using the following primer set in PCR (35 cycles of 10 seconds at 98° C., 5 seconds at 55° C., and 15 seconds at 72° C.; followed by 5 minutes at 72° C.), and the obtained amplification fragment was cut with BamHI (preparing a BamHI cut product of the PCR fragment of the Z-domain gene).

<Primer set>
SEQ ID NO: 9
5′-GATAACAAATTCAACAAAGAACAAC-3′:
SEQ ID NO: 13
5′-GCGGATCCAGCTTTTGGTGCTTGTGC-3′:

From an expression plasmid created in advance by inserting the SLY-SS(C/A) gene in the BamHI/PstI site of pQE-1, the insertion part was cut out using BamHI and PstI and purified. This was ligated with the BamHI cut product of the PCR fragment of the Z-domain gene for 2 hours at 16° C. This ligation solution was used as a template for amplification using the following primer set in PCR (35 cycles of 10 seconds at 98° C., 5 seconds at 55° C., and 15 seconds at 72° C.; followed by 5 minutes at 72° C.)

<Primer set>
SEQ ID NO: 9
5′-GATAACAAATTCAACAAAGAACAAC-3′:
SEQ ID NO: 14
5′-CGCTGCAGTTACTCTATCACCTC-3′:

The obtained fragment was treated with PstI, and the obtained cut product was inserted and ligated in the part of pQE-1 vector cut by PvuII and PstI. This was used to transform strain DH5-alpha Z1 of Escherichia coli (a gift from Dr. Bernd Bukau of University of Heidelberg) to obtain an expression strain of the CDC variant (Z-SLY-SS(C/A)) having the Z-domain fused to the N-terminal side.

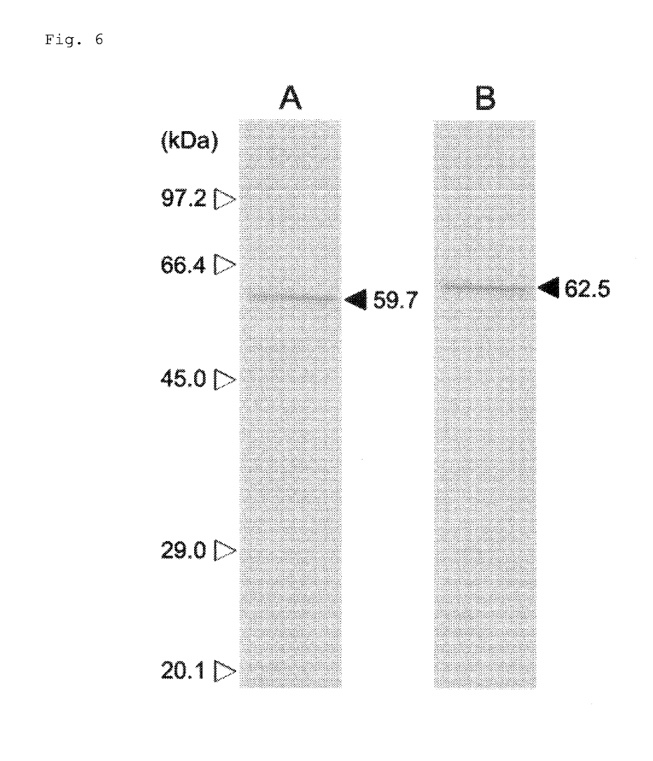

Escherichia coli for expressing Z-SLY-SS(C/A) encoding the amino acid sequence (FIG. 5A; SEQ ID NO: 7) of Z-SLY-SS(C/A) was cultured in LB medium at a large scale (2 L), harvested using a centrifuge, and sonicated to obtain a crude fraction of the CDC variant (Z-SLY-SS(C/A)) from bacterial cells. Through chelate affinity chromatography using AKTAprime plus (GE healthcare) mounted with a HisTrap HP column, the CDC variant (Z-SLY-SS(C/A)) was purified from the crude fraction, and the main fraction was dialyzed against PBS to obtain a purified preparation (molecular weight: 59.7 kDa). SDS-PAGE was conducted using the preparation, and CBB staining was performed thereon to examine the purity of the preparation (FIG. 6A). The preparation was cryopreserved at −80° C. until use.

### (2-2) Creation of Z-cSLY-SS(C/A)

By using, as a template, a plasmid obtained by cloning ILY-SS created from ily gene of strain UNS46 of S. intermedius (a gift from Dr. Robert A. Whiley of Bart's and The London School of Medicine and Dentistry) in BamHI/PstI site of pQE-1 vector, PCR was performed using the following primer set. As a result, gene regions of domains 1 to 3 of ILY-SS including, at one end, a BamHI recognition cleavage site, and, at the other end, 15 bases encoding 5 amino acid residues from the N-terminal side of domain 4 of SLY was amplified.

<Primer set>
SEQ ID NO: 15
5′-CGGGATCCGAAACACCTACCAAACC-3′:
SEQ ID NO: 16
5′-CAATGTCAATGCACTATCTTTATAGGATGTTAC-3′:

Next, by using, as a template, an expression plasmid created in advance having the SLY-SS(C/A) gene inserted in BamHI/PstI site of pQE-1, PCR was performed using the following primer set. As a result, gene regions of domain 4 of SLY-SS(C/A) including, at one end, 15 bases encoding 5 amino acid residues from the C-terminal side of domains 1 to 3 of ILY, and, at the other end, a PstI recognition cleavage site was amplified.

<Primer set>
SEQ ID NO: 17
5′-ACATCCTATAAAGATAGTGCATTGACATTG-3′:
SEQ ID NO: 14
5′-CGCTGCAGTTACTCTATCACCTC-3′:

The two amplification fragments were applied in agarose gel electrophoresis, cut out therefrom, purified, mixed, and used in fusing PCR with a primer set shown in the following to obtain a gene of a chimeric CDC variant (cSLY-SS(C/A)) which is a product of fusing region of domain 4 of SLY-SS(C/A) and regions of domain 1 to 3 of ILY-SS having recognition cleavage sites of BamHI and PstI at the ends.

<Primer set>
SEQ ID NO; 15
5′-CGGGATCCGAAACACCTACCAAACC-3′:
SEQ ID NO: 14
5′-CGCTGCAGTTACTCTATCACCTC-3′:

Next, the gene fragment of the chimeric CDC variant (cSLY-SS(C/A)) was cut using BamHI and PstI, and this cSLY-SS(C/A) gene fragment was inserted, as a substitute, in the BamHI/PstI site of the Z-SLY-SS(C/A) expression plasmid created in advance. The obtained plasmid was used to transform Escherichia coli, and an expression system of the chimeric CDC variant (Z-cSLY-SS(C/A)) was established.

Escherichia coli for expressing Z-cSLY-SS(C/A) encoding the amino acid sequence (FIG. 5B; SEQ ID NO: 8) of chimeric CDC variant (Z-cSLY-SS(C/A)) was cultured in LB medium at a large scale (2 L), harvested using a centrifuge, and sonicated to obtain a crude fraction of the chimeric CDC variant (Z-cSLY-SS(C/A)) from bacterial cells. Through chelate affinity chromatography using AKTAprime plus (GE healthcare) mounted with a HisTrap HP column, purification was performed on the crude fraction, and the main fraction was dialyzed against PBS to obtain a purified preparation (molecular weight; 62.5 kDa). SDS-PAGE was conducted using the preparation, and CBB staining was performed thereon to examine the purity of the preparation (FIG. 6B). The preparation was cryopreserved at −80° C. until use.

### Example 2

### Functional Evaluation of CDC Variant (Z-CDC-SS)—Membrane Pore Forming Activity of Z-CDC-SS

Membrane pore forming activities of the CDC variants (Z-SLY-SS(C/A), Z-cSLY-SS(C/A)) created in Example 1 were evaluated by measuring their hemolysis activity against human red blood cells.

Red blood cells prepared by centrifuging blood obtained from healthy volunteers were rinsed in phosphate buffered saline (PBS), and a PBS suspension containing 25% red blood cells was prepared. The red blood cell suspension was added in reaction solutions (PBS) such that the concentration of red blood cells became 0.5%, and CDC variants were each added thereto and were allowed to react for 1 hour at 37° C. In order to observe the difference in hemolysis activity when the intermolecular SS bond was cleaved and un-cleaved, the experiment was conducted under two conditions of 10 mM dithiothreitol (DTT) existing condition (under reducing condition) and DTT non-existing condition (under nonreducing condition). Then, the reaction mixtures were centrifuged, and 200 μl from each of the obtained supernatants was dispensed in a 96-well microplate to measure the 540 nm absorbance using a microplate reader (Bio-Rad Model 550). A measured value obtained when the red blood cells were treated with PBS was defined as hemolysis activity of 0% (negative control), whereas a measured value obtained when the red blood cells were treated with purified water was defined as hemolysis activity of 100% (positive control), and hemolysis activities, i.e., membrane pore forming activities, of each of the CDC variants were calculated.

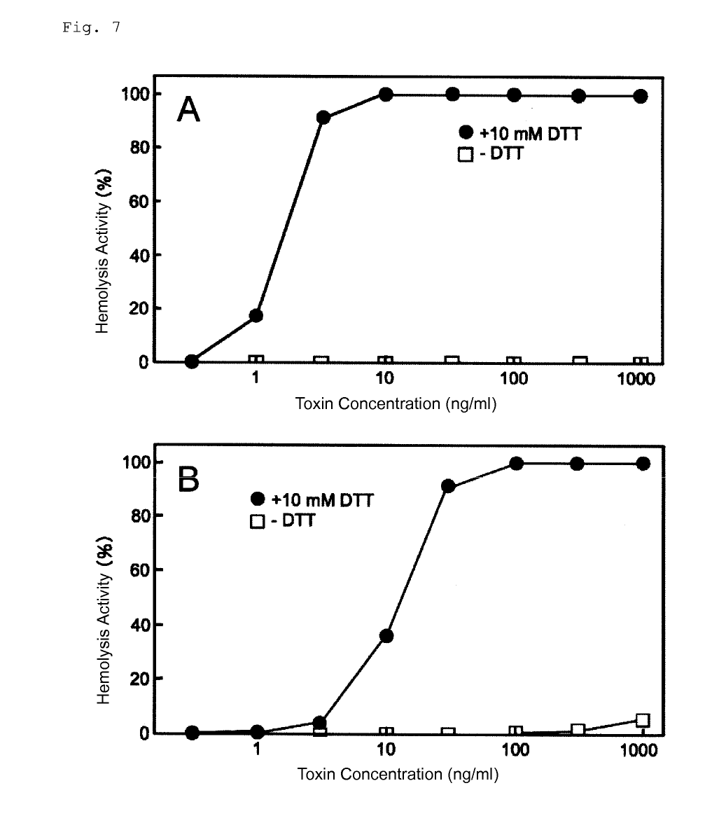

The hemolysis activities exhibited by Z-SLY-SS(C/A) and Z-cSLY-SS(C/A) each under DTT existing condition (under reducing condition: dots connected by a line) and under DTT non-existing condition (under nonreducing condition: open squares connected by a line) are shown in A and B of FIG. 7.

As shown in FIG. 7, the two types of CDC variants (Z-SLY-SS(C/A), Z-cSLY-SS(C/A)) created in Example 1 did not exhibit hemolysis activities (membrane pore forming activities) under nonreducing condition not causing cleavage of an intermolecular SS bond at all, and exhibited hemolysis activities (membrane pore forming activities) only under reducing condition causing cleavage of an SS bond. In addition, the concentration of each toxin exhibiting 50% hemolysis activity was 1.7 ng/ml in the Z-SLY-SS(C/A) variant and 12.4 ng/ml in the Z-cSLY-SS(C/A) variant. This revealed that membrane pores are formed at extremely low concentration in each of the CDC variants.

With the results above, it was confirmed that the CDC variants of the present invention can form a membrane pore selectively on a cell membrane or a cholesterol-containing membrane under reducing condition causing cleavage of an SS bond, and can release (elute) a substance contained therein.

### Example 3

### Functional Evaluation of CDC Variant (Z-CDC-SS)—Antibody Binding Activity of Z-CDC-SS

IgG binding activities of the CDC variants (Z-SLY-SS(C/A), Z-cSLY-SS(C/A)) having the Z-domain were evaluated in the following manner.

First, in each well of a 96-well microplate (ELISA plate, IWAKI), 1 μg each of IgG purified from normal serum of a healthy volunteer or normal serum of rabbit was dried, fixed, and blocked for 30 minutes in PBS containing 1% bovine serum albumin (blocking solution). Next, a dilution series was prepared with the blocking solution for each of the CDC variants (Z-SLY-SS(C/A), Z-cSLY-SS(C/A)) or Z-domain deletion variants (LTBP-SLY-SS(C/A), LTBP-cSLY-SS(C/A)) obtained by substituting the Z-domain (SEQ ID NO: 6) of each of the CDC variants (Z-SLY-SS(C/A), Z-cSLY-SS(C/A)) with a LTBP peptide consisting of 20 amino acid residues (SEQ ID NO: 18) that does not have affinity with the antibody. The dilution series were added to the wells and were allowed to react for 1 hour. Then, the wells were rinsed 6 times with PBS, and a hybridoma culture solution containing anti-SLY mouse monoclonal antibody (an IgG1 antibody that was produced by the present inventors with Balb/c mice using purified SLY as an antigen, and whose epitope is domain 4 of SLY) was added thereto and allowed to react for 1 hour. The wells were rinsed 6 times with PBS, and a horseradish peroxidase (HRP) labeled anti-mouse IgG goat antibody (acquired from Kirkegaard & Perry Laboratories) diluted in the blocking solution was added thereto and allowed to react for 1 hour. After the reaction, the wells were rinsed 6 times again with PBS. Lastly, an HRP substrate solution of a 50 mM sodium phosphate buffer (pH 4.5) containing 2 mM 2,2′-azino-di-(3-ethyl-benzothiazoline-6-sulfonic acid) (ABTS) and 0.002% hydrogen peroxide was dispensed in each of the wells and allowed to react for a predetermined period of time. Absorbance of the wells at 415 nm was measured using a microplate reader.

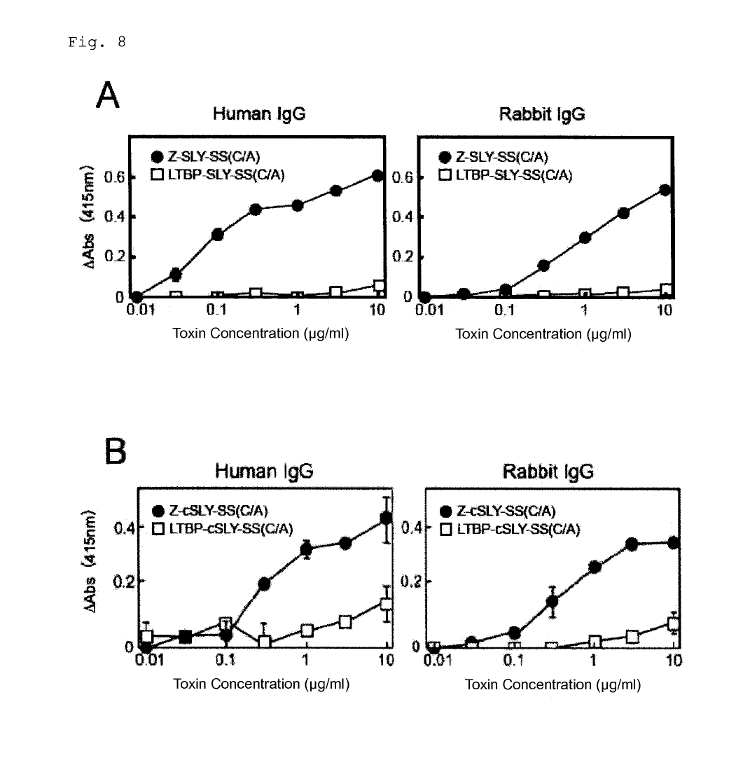

The results are shown in FIG. 8. The left graph and the right graph in A of FIG. 8 respectively show the results of binding activities against human IgG and rabbit IgG for Z-SLY-SS(C/A) (dots connected by a line) having the Z-domain and LTBP-SLY-SS(C/A) (open squares connected by a line) lacking the Z-domain. The left graph and the right graph in B of FIG. 8 respectively show the results of binding activities against human IgG and rabbit IgG for Z-cSLY-SS(C/A) (dots connected by a line) having the Z-domain and LTBP-cSLY-SS(C/A) (open squares connected by a line) lacking the Z-domain.

As can be understood from the results, the CDC variants (Z-SLY-SS(C/A) and Z-cSLY-SS(C/A)) of the present invention having the Z-domain both exhibited IgG binding activity in a concentration-dependent manner, whereas the Z-domain deletion variants (LTBP-SLY-SS(C/A) and LTBP-cSLY-SS(C/A)) obtained therefrom by removing the Z-domain both did not show IgG binding activity. From this, it was confirmed that the CDC variants (Z-SLY-SS(C/A) and Z-cSLY-SS(C/A)) of the present invention created in Example 1 retain the activity to bind with antibodies (IgG) from human or rabbit via their Z-domain.

### Example 4

### Targeting of Liposome to CEA Positive Cancer Cells Using Z-cSLY-SS(C/A) and Anti-CEA Antibody

### (1) Creation of Medical Agent-Encapsulating Liposome (Mock Liposome)

In order to establish a visualization model of the drug delivery system (DDS), as a medical agent-encapsulating liposome (mock liposome) to be delivered to a target site, a 1,2-dipalmitoyl-sn-glycero-3-phosphatidylcholine (hereinafter, referred to as “DPPC”)/cholesterol (1:1) liposome encapsulating 10 mM fluorescent dye uranine (fluorescein Na) was prepared.

Specifically, by using a hitherto known method, DPPC and cholesterol were dissolved in chloroform in a proportion of 1:1 (mole ratio). The mixture was applied in an eggplant type flask as a coating under reduced pressure, and dried. PBS containing 10 mM uranine was added thereto, and ultrasonic treatment was performed thereon in an ultrasonic-wave tank at 60° C. to create vesicles. This was frozen and thawed repeatedly for 5 times using liquid nitrogen and a 60° C. temperature controlled bath, and caused to pass through a membrane filter, whose pore diameter was 100 nm, 21 times using a Mini-extruder (Avanti) to create ULM liposome.

This was passed through a SephadexG50 gel filtration column to collect an uranine-encapsulating liposome fraction that did not contain unencapsulated uranine. In addition, condensing of that using an ultra-centrifuge was conducted in accordance with need, and the fraction was preserved at 4° C. until use.

For the purpose of confirming binding between the thus prepared uranine-encapsulating liposome fraction and the CDC variant (Z-SLY-SS(C/A) or Z-cSLY-SS(C/A)) of the present invention, a certain amount of each of the CDC variants (Z-SLY-SS(C/A), Z-cSLY-SS(C/A)) was allowed to react with the uranine-encapsulating liposome, and the mixture was ultra-centrifuged. Hemolysis activity of supernatant collected therefrom was measured. In addition, as a positive control, a certain amount of each of the CDC variants (Z-SLY-SS(C/A), Z-cSLY-SS(C/A)) was allowed to react without having the uranine-encapsulating liposome added thereto, and ultra-centrifuged. Hemolysis activity of supernatant collected therefrom was measured. It should be noted that measuring of hemolysis activity was conducted under reducing condition with 10 mM DTT, in accordance with the method set forth in Example 2.

In the result, the hemolysis activity of the supernatant collected after reaction with the uranine-encapsulating liposome was less than 1% of the hemolysis activity of the supernatant collected without reaction with the uranine-encapsulating liposome. Therefore, it was revealed that 99% or more of both the CDC variants (Z-SLY-SS(C/A), Z-cSLY-SS(C/A)) of the present invention were bound to the uranine-encapsulating liposome.

(2) Creation of Drug Delivery System and Evaluation Thereof

A drug delivery system was created using the above described uranine-encapsulating liposome as a medical agent-encapsulating liposome (mock liposome).

Specifically, the uranine-encapsulating liposome fraction and each of the CDC variants (Z-SLY-SS(C/A), Z-cSLY-SS(C/A)) were mixed such that mole ratio thereof with respect to DPPC in the liposome was 400:1 (DPPC:CDC variant). The mixture was allowed to react at 25° C. for 30 minutes to bind the CDC variant and the uranine-encapsulating liposome. The mixture was allowed to react with an antibody (anti-CEA rabbit IgG antibody (ABBIOTEC LLC.)) (hereinafter, referred to as “anti-CEA antibody”) against CEA which is a carcinoembryonic antigen. After rinsing, its supernatant was replaced with a DMEM medium containing 10% fetal bovine serum. The product was re-suspended to be the same concentration to produce a drug delivery system bound with anti-CEA antibody (anti-CEA antibody—[Z-SLY-SS(C/A)]—uranine encapsulating liposome, anti-CEA antibody—[Z-cSLY-SS(C/A)]—uranine encapsulating liposome).

The obtained product was added on a collagen coated cover glass having cultured thereon a mixture of cells including CEA positive human colon cancer cells (Lovo cell) expressing CEA and, as control cells, human normal fibroblast (NB1RGB cell), and the cover glass was placed in a carbon dioxide gas incubator to allow reaction at 37° C. for 2 hours. As a control, a liposome fraction (uranine-encapsulating liposome fraction) that was not bound to either Z-SLY-SS(C/A) or Z-cSLY-SS(C/A) and prepared to have the same concentration was also allowed to react similarly and had the following operation performed thereon.

After the reaction, the supernatant was removed, and the cover glass was rinsed three times using a DMEM medium that did not contain serum and was observed in an inverted fluorescence microscope IX71 (Olympus).

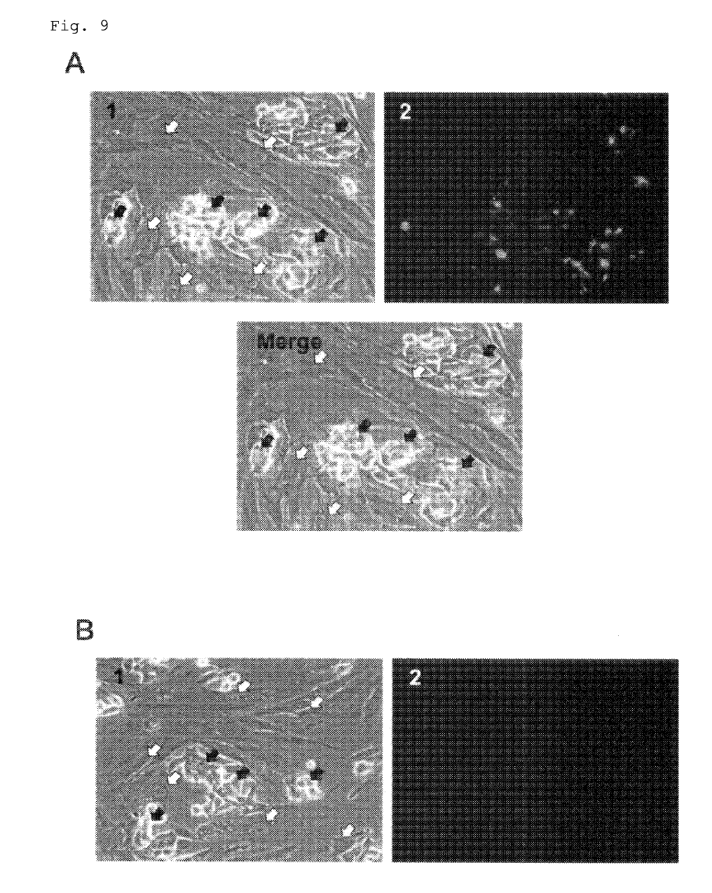

As a representative example, the results obtained from the anti-CEA antibody—[Z-cSLY-SS(C/A)]—uranine encapsulating liposome and control are shown in FIG. 9. FIG. 9A shows the result of treating cells with anti-CEA antibody—[Z-cSLY-SS(C/A)]—uranine encapsulating liposomes, and FIG. 9B shows the result of treating cells with uranine-encapsulating liposomes. In each figure, “1” is an image of cells observed with a phase contrast microscope, “2” is an uranine fluorescence image observed with the inverted fluorescence microscope, and “Merge” (only in A) is an overlay of the images of 1 and 2. Black arrows indicate CEA positive human colon cancer cells (Lovo cells), and white arrows indicate human normal fibroblast (NB1RGB cells).

As can be understood from the figure, the anti-CEA antibody—[Z-cSLY-SS(C/A)]—uranine encapsulating liposome was observed binding specifically to CEA positive cells (Lovo cell) as a target even in a mixture of normal cells and CEA positive cells. Although there were some cells with the liposome remaining on the cell surface, in the image, fluorescent dye of uranine was observed to be intracellularly taken in and diffused in some of the cells. On the other hand, the liposome that was not bound to the CDC variant showed little affinity against any of the cells (normal cells and CEA positive cells (Lovo cell)), and was confirmed to be easily removed through a rinsing operation.

From this, it was confirmed that, with the drug delivery system utilizing, as a carrier, the CDC variants (Z-SLY-SS(C/A), Z-cSLY-SS(C/A)) of the present invention created in Example 1, a liposome encapsulating an intended drug can be delivered to a target cell based on the specificity of the antibody bound to the targeting part, and can cause, after reaching the target cell, the drug in the liposome to be taken in the target cell to allow the drug to take effect.

From the results described above, it was confirmed that, by binding the CDC variants (Z-SLY-SS(C/A), Z-cSLY-SS(C/A)) of the present invention created in Example 1 to an antibody, a cholesterol-containing liposome having a size of 10 nm to 300 nm in diameter encapsulating a desired substance such as a drug, preferably a liposome having a size of 30 nm to 100 nm, is useful as a DDS carrier (drug carrier) that is to be delivered to a target cell and taken into the cell.

### Example 5

### Evaluation of Delivery and Anticancer Action of DDS in Cancer-Bearing Nude Mouse

### 1. Preparation of Medical Agent-Encapsulating Liposome

By using 5 mM 5-fluorouracil (5-FU) as a medical agent to be delivered and 10 mM fluorescent dye uranine (fluorescein Na) as a visualization agent for evaluating sealing ability of liposome, a dipalmitoylphosphatidylcholine (DPPC)/cholesterol (1:1) liposome was prepared with Bangham method.

Specifically, by using a hitherto known method, 10 μmol of DPPC and 10 μmol of cholesterol were dissolved in chloroform. The mixture was applied in an eggplant type flask as a coating under reduced pressure, and dried. 1 ml of phosphate buffered saline (PBS) containing 5 mM 5-FU and 10 mM uranine was added thereto, and ultrasonic treatment was performed thereon in an ultrasonic-wave tank at 60° C. to create vesicles. This was frozen and thawed repeatedly for 5 times using liquid nitrogen and a 60° C. temperature controlled bath, and caused to pass through a 100 nm membrane filter using a Mini-extruder (Aventi) to create ULM liposome. This was passed through a SephadexG50 gel filtration column to collect an uranine-encapsulating liposome fraction that did not contain unencapsulated uranine. The liposome prepared in such manner was used in the following experiment as a medical agent (5-FU)-encapsulating liposome.

When the phospholipid concentration of the liposome at the time of usage was measured using a phospholipid quantification kit (phospholipid C-Test Wako), the phospholipid concentration of the liposome was 25 μM.

2. Preparation of DDS (Medical Agent Supporting—Antibody Bound CDC Variant)

The 5-FU-containing liposome (equivalent to 75 nmol of DPPC) prepared above was processed for 1 hour at 25° C. and bound with 36 μg of the CDC variant (Z-cSLY-SS(C/A)). Next, the mixture was treated with 10 μM cholesterol-containing PBS to conduct a neutralization process of masking the potentially-included unbound cholesterol-binding domain of Z-cSLY-SS(C/A). Furthermore, 36 μg of anti-carcinoembryonic antigen (CEA) antibody (IgG) was added thereto and allowed to react for linking the CDC variant and the antibody to prepare an antibody bound CDC variant supporting 5-FU as a medical agent (5-FU-supporting antibody-bound CDC variant). This was used as the DDS for the following animal experiment.

In addition, for a comparative experiment, the 5-FU-containing liposome (equivalent to 75 nmol of DPPC) prepared above was processed for 1 hour at 25° C. and bound with 36 μg of the CDC variant (Z-cSLY-SS(C/A)). With this, a “5-FU-supporting CDC variant” that did not have an antibody bound thereto was created, and this was used in the following animal experiment.

3. DDS Administration to Cancer-Bearing Nude Mouse and Evaluation of Anticancer Action

Balb/cSlc-nu/nu mice (male, 11 weeks of age) were used as nude mice, and the mice were randomly separated in to 4 groups (5 in each group). Among these groups, mice in groups 1 to 3 were transplanted intraperitoneally with 1.0×10⁶ CEA positive human hepatic-cancer cells HepG2 suspended in 1 ml of penicillin/streptomycin-containing DMEM medium. On the next day, mice in each of the groups were intraperitoneally administered with PBS or the following PBS suspension.

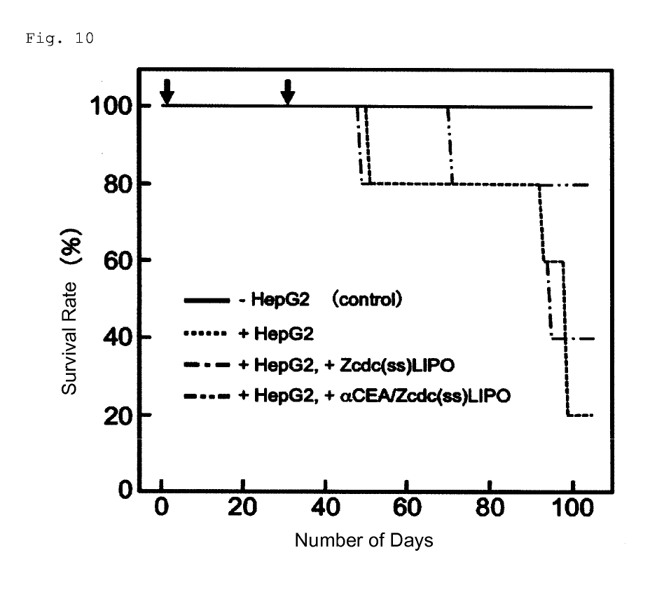

(1) Group 1: 0.1 ml of a PBS suspension obtained by suspending “5-FU-supporting antibody-bound CDC variant” prepared above in PBS (in FIG. 10, labeled as +HepG2, +alpha-CEA/Zcdc(ss) LIPO) was intraperitoneally administered.
(2) Group 2: 0.1 ml of a PBS suspension obtained by suspending “5-FU-supporting CDC variant” prepared above in PBS (in FIG. 10, labeled as +HepG2, +Zcdc(ss) LIPO) was intraperitoneally administered.
(3) Group 3: 0.1 ml of PBS (in FIG. 10, labeled as +HepG2) was intraperitoneally administered.

Mice in group 4, which is the control group, were not transplanted with human hepatic-cancer cells HepG2, and when the mice in the other groups were administered with PBS or the PBS suspension described above, they were administered with 0.1 ml of PBS at the same time (in FIG. 10, labeled as −HepG2 (control)).

30 days after the administration (first administration) of the PBS suspension or PBS, mice in each of the groups were intraperitoneally administered with the same PBS suspension or PBS. However, this time, although the “5-FU-supporting antibody-bound CDC variant” and “5-FU-supporting CDC variant” administered to group 1 and group 2 had the same amount of liposome (5-FU amount) administered the first time, the amount of the CDC variant (Z-cSLY-SS(C/A)) and anti-CEA antibody (IgG) were both reduced to ⅔ of the amount.

Then, while individually measuring weight and observing status of the mice in each of the groups, their survival were monitored.

FIG. 10 shows survival rate in each of the groups during the course of time. From this result, it was confirmed that administration of the DDS of the present invention markedly increases survival rate, since, while the 100-th day survival rate of the cancer-bearing mice (group 3) not treated with DDS was 20%, the 100-th day survival rate of the cancer-bearing mice (group 2) administered with the “5-FU-supporting CDC variant” was 40% and the 100-th day survival rate of the cancer-bearing mice (group 1) administered with the “5-FU-supporting antibody-bound CDC variant” was 80%. From this, it is thought that the DDS (drug supporting—antibody bound CDC variant) of the present invention was transferred and delivered selectively to a target cell, and effectively exerted its medicinal effect at the site.

Therefore, it was confirmed that the CDC variant of the present invention, when bound to a desired antibody and drug, can selectively and effectively transport the drug to a desired target cell, and can allow its medicinal effect to be effectively exerted within the cell.

Sequence Listing Free Text

As one example of Z-SLY-SS(C/X), SEQ ID NO: 7 shows the amino acid sequence of Z-SLY-SS(C/A) (FIG. 5A) created in Example 1. As one example of Z-cSLY-SS(C/X), SEQ ID NO: 8 shows the amino acid sequence of Z-cSLY-SS(C/A) (FIG. 5B) created in Example 1. SEQ ID NOS: 9, 10, and 13 to 17 show base sequences of primers, and SEQ ID NO: 11 shows the amino acid sequence of the hexa His tag.

1. A cholesterol-dependent cytolysin variant comprising:
(1) an antibody binding domain;
(2) modified domains 1 to 3 wherein at least two arbitrary amino acid residues in domains 1 to 3 of cholesterol-dependent cytolysin are substituted with Cys residues to form an SS bond with each other under nonreducing condition; and
(3) a modified domain 4 wherein all Cys residues in domain 4 of cholesterol-dependent cytolysin are substituted with an amino acid residue selected from the group consisting of Ala, Ser, Gly, and Thr,
wherein the cholesterol-dependent cytolysin variant has an ability to bind with a cell membrane or a cholesterol-containing liposomal membrane, and exerts an ability to form a membrane pore when an SS bond is cleaved under reducing condition.

2. The cholesterol-dependent cytolysin variant according to claim 1, wherein the cholesterol-dependent cytolysin is at least one type selected from suilysin and intermedilysin.

3. The cholesterol-dependent cytolysin variant according to claim 1, wherein domains 1 to 3 and domain 4 of the cholesterol-dependent cytolysin are all derived from suilysin, or domains 1 to 3 of the cholesterol-dependent cytolysin are derived from intermedilysin and domain 4 of the cholesterol-dependent cytolysin is derived from suilysin.

4. The cholesterol-dependent cytolysin variant according to claim 1, wherein the antibody binding domain is Z-domain of Protein A from Staphylococcus aureus, or B-domain of Protein G derived from group G streptococcus.

5. The cholesterol-dependent cytolysin variant according to claim 1, wherein the reducing condition is a reducing environment generated by intracellular glutathione.

6. The cholesterol-dependent cytolysin variant according to claim 5, wherein the reducing environment generated by the intracellular glutathione is an environment within a cytoplasm or a phagolysosome containing 1 to 10 mM of reduced glutathione.

7. A drug carrier comprising the cholesterol-dependent cytolysin variant according to claim 1 and a cell or tissue specific antibody bound thereto via the antibody binding domain.

8. The drug carrier according to claim 7, wherein the cell or tissue specific antibody is an antibody specifically recognizing an oligosaccharide or protein specifically expressed in a cancer cell, an antibody specifically recognizing a viral protein that appears on a cell membrane of a virally infected cell, or an antibody specifically recognizing an CD antigen expressed specifically on an immune cell.

9. A drug delivery system comprising the drug carrier according to claim 7, the drug carrier having bound thereto, via domain 4 of the cholesterol-dependent cytolysin variant, a cholesterol-containing microcapsule filled with a medicinal ingredient or a bioactive substance, or a functional cell.

10. The drug delivery system according to claim 9, wherein the functional cell is an immune cell or a recombinant cell.

11. The drug delivery system according to claim 9, wherein the medicinal ingredient or the bioactive substance is at least one selected from the group consisting of compounds, peptides, proteins, nucleic acids and substances with a dominant negative effect, having medicinal effects or being bioactive.

12. The cholesterol-dependent cytolysin variant according to claim 2, wherein domains 1 to 3 and domain 4 of the cholesterol-dependent cytolysin are all derived from suilysin, or domains 1 to 3 of the cholesterol-dependent cytolysin are derived from intermedilysin and domain 4 of the cholesterol-dependent cytolysin is derived from suilysin.

13. The cholesterol-dependent cytolysin variant according to claim 2, wherein the antibody binding domain is Z-domain of Protein A from Staphylococcus aureus, or B-domain of Protein G derived from group G streptococcus.

14. The cholesterol-dependent cytolysin variant according to claim 3, wherein the antibody binding domain is Z-domain of Protein A from Staphylococcus aureus, or B-domain of Protein G derived from group G streptococcus.

15. The cholesterol-dependent cytolysin variant according to claim 2, wherein the reducing condition is a reducing environment generated by intracellular glutathione.

16. The cholesterol-dependent cytolysin variant according to claim 3, wherein the reducing condition is a reducing environment generated by intracellular glutathione.

17. The cholesterol-dependent cytolysin variant according to claim 4, wherein the reducing condition is a reducing environment generated by intracellular glutathione.

18. A drug carrier comprising the cholesterol-dependent cytolysin variant according to claim 2 and a cell or tissue specific antibody bound thereto via the antibody binding domain.

19. A drug carrier comprising the cholesterol-dependent cytolysin variant according to claim 3 and a cell or tissue specific antibody bound thereto via the antibody binding domain.

20. A drug carrier comprising the cholesterol-dependent cytolysin variant according to claim 4 and a cell or tissue specific antibody bound thereto via the antibody binding domain.
